SEQUENCE LISTING (72 sequences)

SEQ ID NO: 1 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 2 - protein, 261 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 3 - protein, 10 aa, Homo sapiens
DQWSTQDLYN

SEQ ID NO: 4 - protein, 11 aa, Homo sapiens
NNPVTAVFNYQ

SEQ ID NO: 5 - protein, 14 aa, Homo sapiens
STQDLYNNPVTAVF

SEQ ID NO: 6 - protein, 13 aa, Homo sapiens
TNFWMSTANMYTG

SEQ ID NO: 7 - protein, 13 aa, Homo sapiens
DSAKANMTLTSGI

SEQ ID NO: 8 - protein, 55 aa, Homo sapiens
MDQWSTQDLYNNPVTAVFNYQGLWRSCVRESSGFTECRGYFTLLGLPAMLQAVRA

SEQ ID NO: 9 - protein, 24 aa, Homo sapiens
FALKCIRIGSMEDSAKANMTLTSG

SEQ ID NO: 10 - protein, 40 aa, Homo sapiens
ANMLVTNFWMSTANMYTGMGGMVQTVQTRYTFGAALFVGW

SEQ ID NO: 11 - protein, 153 aa, Homo sapiens
MDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGW

SEQ ID NO: 12 - protein, 107 aa, Artificial [sequence omitted]

SEQ ID NO: 13 - protein, 326 aa, Artificial
  Description of artificial sequence Translation of PCR product
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 466 aa, Artificial
  Description of artificial sequence chimeric monoclonal antibody
MEWTWVFLFLLSVTAGVHSQVQLQQSGAELMKPGASVKISCKATGYTFSSYWIEWVKQRPGHGLEWIGEILPGSGSTNYNEKFKGKATFTADTSSNTAYMQLSSLTSEDSAVYYCARYDYPWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 467 aa, Artificial
  Description of artificial sequence chimeric monoclonal antibody
MDWLWNLLFLMAAAQSIQAQIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 465 aa, Artificial
  Description of artificial sequence chimeric monoclonal antibody
MEWIWIFLFILSGTAGVHSQVQLQQSGAELARPGASVKLSCKASGYTFTDYYINWVKQRTGQGLEWIGEIYPGSGNTYYNEKFKGKATLTADKSSSTAYMQLSSLTSEDSAVYFCARSYGAFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 467 aa, Artificial
  Description of artificial sequence chimeric monoclonal antibody
MGWSCIILFLVATATGVHSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 466 aa, Artificial
  Description of artificial sequence chimeric monoclonal antibody
MEWRIFLFILSGTAGVHSQVQLQQSGPELVKPGASVKMSCKASGYTFTDYVISWVKQRTGQGLEWIGEIYPGSGSTYYNEKFKGKATLTADKSSNTAYMQLSSLTSEDSAVYFCARGVLLRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 469 aa, Artificial [sequence omitted]

SEQ ID NO: 20 - protein, 240 aa, Artificial
  Description of artificial sequence chimeric monoclonal antibody
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 235 aa, Artificial
  Description of artificial sequence chimeric monoclonal antibody
MHFQVQIFSFLLISASVIMSRGQIVLTQSPAIMSASPGEKVTITCSASSSVSYMHWFQQKPGTSPKLWIYSTSNLASGVPARFSGSGSGTSYSLTISRMEAEDAATYYCQQRSSYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 234 aa, Artificial
  Description of artificial sequence chimeric monoclonal antibody
MEFQTQVFVFVLLWLSGVDGDIVMTQSQKFMSTSVGDRVSITCKASQNVRTAVAWYQQKPGQSPKALIYLASNRHTGVPDRFTGSGSGTDFTLTISNVQSEDLADYFCLQHWNYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 240 aa, Artificial
  Description of artificial sequence chimeric monoclonal antibody
MDSQAQVLMLLLLWVSGTCGDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 240 aa, Artificial
  Description of artificial sequence chimeric monoclonal antibody
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 239 aa, Artificial
  Description of artificial sequence chimeric monoclonal antibody
MDSQAQVLILLLLWVSGTCGDIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 240 aa, Artificial
  Description of artificial sequence chimeric monoclonal antibody
MDSQAQVLMLLLLWVSGTCGDIVMSQSPSSLAVSVGEKVTMSCKSSQSLLYSSNQKNYLAWYQQKPGQSPKLLIYWASTRESGVPDRFTGSGSATDFTLTISSVQAEDLADYHCGQGYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 240 aa, Artificial; identical to SEQ ID NO: 23
  Description of artificial sequence chimeric monoclonal antibody

SEQ ID NO: 28 - protein, 234 aa, Artificial
  Description of artificial sequence chimeric monoclonal antibody
MESQTLVFISILLWLYGADGNIVMTQSPKSMSMSVGERVTLTCKASENVVTYVSWYQQKPEQSPKLLIYGASNRYTGVPDRFTGSGSATDFTLTISSVKAEDLAVYYCQQYYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 117 aa, Artificial
  Description of artificial sequence Translation of PCR product
QVQLQQSGAELMKPGASVKISCKATGYTFSSYWIEWVKQRPGHGLEWIGEILPGSGSTNYNEKFKGKATFTADTSSNTAYMQLSSLTSEDSAVYYCARYDYPWFAYWGQGTLVTVSA

SEQ ID NO: 30 - protein, 118 aa, Artificial
  Description of artificial sequence Translation of PCR product
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSS

SEQ ID NO: 31 - protein, 116 aa, Artificial
  Description of artificial sequence Translation of PCR product
QVQLQQSGAELARPGASVKLSCKASGYTFTDYYINWVKQRTGQGLEWIGEIYPGSGNTYYNEKFKGKATLTADKSSSTAYMQLSSLTSEDSAVYFCARSYGAFDYWGQGTTLTVSS

SEQ ID NO: 32 - protein, 118 aa, Artificial
  Description of artificial sequence Translation of PCR product
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 33 - protein, 118 aa, Artificial
  Description of artificial sequence Translation of PCR product
QVQLQQSGPELVKPGASVKMSCKASGYTFTDYVISWVKQRTGQGLEWIGEIYPGSGSTYYNEKFKGKATLTADKSSNTAYMQLSSLTSEDSAVYFCARGVLLRAMDYWGQGTSVTVSS

SEQ ID NO: 34 - protein, 120 aa, Artificial
  Description of artificial sequence Translation of PCR product
QVHLQQSGSELRSPGSSVKLSCKDFDSEVFPFAYMSWIRQKPGHGFEWIGDILPSIGRTIYGEKFEDKATLDADTVSNTAYLELNSLTSEDSAIYYCARGEGYGAWFAYWGQGTLVTVSA

SEQ ID NO: 35 - protein, 113 aa, Artificial
  Description of artificial sequence Translation of PCR product
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 36 - protein, 106 aa, Artificial
  Description of artificial sequence Translation of PCR product
QIVLTQSPAIMSASPGEKVTITCSASSSVSYMHWFQQKPGTSPKLWIYSTSNLASGVPARFSGSGSGTSYSLTISRMEAEDAATYYCQQRSSYPPTFGGGTKLEIK

SEQ ID NO: 37 - protein, 107 aa, Artificial
  Description of artificial sequence Translation of PCR product
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPLTFGGGTKLEIK

SEQ ID NO: 38 - protein, 113 aa, Artificial
  Description of artificial sequence Translation of PCR product
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPLTFGAGTKLELK

SEQ ID NO: 39 - protein, 113 aa, Artificial
  Description of artificial sequence Translation of PCR product
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 40 - protein, 112 aa, Artificial
  Description of artificial sequence Translation of PCR product
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLYTFGGGTKLEIK

SEQ ID NO: 41 - protein, 113 aa, Artificial
  Description of artificial sequence Translation of PCR product
DIVMSQSPSSLAVSVGEKVTMSCKSSQSLLYSSNQKNYLAWYQQKPGQSPKLLIYWASTRESGVPDRFTGSGSATDFTLTISSVQAEDLADYHCGQGYSYPYTFGGGTKLEIK

SEQ ID NO: 42 - protein, 113 aa, Artificial; identical to SEQ ID NO: 38
  Description of artificial sequence Translation of PCR product

SEQ ID NO: 43 - protein, 107 aa, Artificial
  Description of artificial sequence Translation of PCR product
NIVMTQSPKSMSMSVGERVTLTCKASENVVTYVSWYQQKPEQSPKLLIYGASNRYTGVPDRFTGSGSATDFTLTISSVKAEDLAVYYCQQYYSYPLTFGAGTKLELK

SEQ ID NO: 44 - protein, 15 aa, Artificial
  Epitope
MDQWSTQDLYNNPVT

SEQ ID NO: 45 - protein, 15 aa, Artificial
  Epitope
STQDLYNNPVTAVFN

SEQ ID NO: 46 - protein, 15 aa, Artificial
  Epitope
LYNNPVTAVFNYQGL

SEQ ID NO: 47 - protein, 15 aa, Artificial
  Epitope
PVTAVFNYQGLWRSC

SEQ ID NO: 48 - protein, 15 aa, Artificial
  Epitope
VFNYQGLWRSCVRES

SEQ ID NO: 49 - protein, 15 aa, Artificial
  Epitope
QGLWRSCVRESSGFT

SEQ ID NO: 50 - protein, 15 aa, Artificial
  Epitope
RSCVRESSGFTECRG

SEQ ID NO: 51 - protein, 467 aa, Artificial
  Description of artificial sequence chimeric monoclonal antibody
MGWSCIILFLVATATGVHSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 326 aa, Artificial
  Description of artificial sequence Translation of PCR product
GPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 447 aa, Artificial Sequence
  Antibody H chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 218 aa, Artificial Sequence
  Antibody L chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 120 aa, Artificial Sequence [sequence omitted]

SEQ ID NO: 56 - protein, 111 aa, Artificial Sequence [sequence omitted]

SEQ ID NO: 57 - protein, 8 aa, Artificial Sequence
  HCDR1
GYTFTNYY

SEQ ID NO: 58 - protein, 8 aa, Artificial Sequence
  HCDR2
INPSNGGT

SEQ ID NO: 59 - protein, 13 aa, Artificial Sequence
  HCDR3
ARRDYRFDMGFDY

SEQ ID NO: 60 - protein, 10 aa, Artificial Sequence
  LCDR1
KGVSTSGYSY

SEQ ID NO: 61 - protein, 3 aa, Artificial Sequence
  LCDR2
LAS

SEQ ID NO: 62 - protein, 9 aa, Artificial Sequence
  LCDR3
QHSRDLPLT

SEQ ID NO: 63 - protein, 440 aa, Artificial Sequence
  Antibody H chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 214 aa, Artificial Sequence
  Antibody L chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 113 aa, Artificial Sequence [sequence omitted]

SEQ ID NO: 66 - protein, 107 aa, Artificial Sequence [sequence omitted]

SEQ ID NO: 67 - protein, 8 aa, Artificial Sequence
  HCDR1
GITFSNSG

SEQ ID NO: 68 - protein, 8 aa, Artificial Sequence
  HCDR2
IWYDGSKR

SEQ ID NO: 69 - protein, 6 aa, Artificial Sequence
  HCDR3
ATNDDY

SEQ ID NO: 70 - protein, 6 aa, Artificial Sequence
  LCDR1
QSVSSY

SEQ ID NO: 71 - protein, 3 aa, Artificial Sequence
  LCDR2
DAS

SEQ ID NO: 72 - protein, 9 aa, Artificial Sequence
  LCDR3
QQSSNWPRT

(19) United States
(12) Patent Application Publication
(10) Pub. No.: US 2024/0199736 A1
(43) Pub. Date: Jun. 20, 2024

(54) COMBINATION THERAPY INVOLVING ANTIBODIES AGAINST CLAUDIN 18.2 FOR TREATMENT OF CANCER

(71) Applicant: ASTELLAS PHARMA INC., Tokyo (JP)

(72) Inventors: Pranob P. BHATTACHARYA, Lake Forest, IL (US); Jane WENG, Tokyo (JP); Fumitaka KINUGASA, Tokyo (JP)

(21) Appl. No.: 18/552,113

(22) PCT Filed: Mar. 24, 2022
(86) PCT No.: PCT/JP2022/017017

§ 371 Date: Sep. 22, 2023

Related U.S. Application Data
(60) Provisional application No. 63/166,019, filed on Mar. 25, 2021.

(51) Int. Cl. C07K 16/28; A61K 31/513; A61K 31/519; A61K 31/555; A61K 31/7068; A61K 39/00; A61P 35/00

(52) U.S. Cl. CPC C07K 16/28; A61K 31/513; A61K 31/519; A61K 31/555; A61K 31/7068; A61P 35/00; A61K 2039/507

(57) ABSTRACT

The present invention provides a combination therapy for treating and/or preventing diseases associated with cells expressing CLDN18.2, including cancer diseases such as gastric cancer, esophageal cancer, pancreatic cancer, lung cancer, ovarian cancer, colon cancer, hepatic cancer, head-neck cancer, and cancer of the gallbladder and metastases thereof.

Cancer of the stomach and the esophagus (gastroesophageal; GE) is one of the malignancies with the highest unmet medical need. Gastric cancer is one of the leading causes of cancer death worldwide. The incidence of esophageal cancer has increased in recent decades, coinciding with a shift in histological type and primary tumor location. Adenocarcinoma of the esophagus is now more prevalent than squamous cell carcinoma in the United States and Western Europe, with most tumors located in the distal esophagus. The overall five-year survival rate for GE cancer is 20-25%, despite the aggressiveness of established standard treatment associated with substantial side effects.

The majority of patients presents with locally advanced or metastatic disease. For these patients, first line treatment is chemotherapy. Treatment regimens are based on a backbone of platinum and fluoropyrimidine derivatives mostly combined with a third compound (e.g., taxane or anthracyclines). Still, median progression free survival of 5 to 7 months and median overall survival of 9 to 11 months are the best that can be expected.

The lack of a major benefit from the various newer generation combination chemotherapy regimens for these cancers has stimulated research into the use of targeted agents. Recently, for Her2/neu-positive gastroesophageal cancers, Trastuzumab has been approved. However, as only ˜20% of patients are eligible for this treatment, the medical need is still high.

The tight junction molecule Claudin 18 splice variant 2 (Claudin 18.2 (CLDN18.2)) is a member of the claudin family of tight junction proteins. CLDN18.2 is a 27.8 kDa transmembrane protein comprising four membrane spanning domains with two small extracellular loops.

In normal tissues there is no detectable expression of CLDN18.2 by RT-PCR with exception of stomach. Immunohistochemistry with CLDN18.2 specific antibodies reveals stomach as the only positive tissue.

CLDN18.2 is a highly selective gastric lineage antigen expressed exclusively on short-lived differentiated gastric epithelial cells. CLDN18.2 is maintained in the course of malignant transformation and thus frequently displayed on the surface of human gastric cancer cells. Moreover, this pan-tumoral antigen is aberrantly expressed at significant levels in esophageal, pancreatic and lung adenocarcinomas. The CLDN18.2 protein is also localized in lymph node metastases of gastric cancer adenocarcinomas and in distant metastases especially into the ovary (so-called Krukenberg tumors) and in liver metastases.

The chimeric IgG1 antibody IMAB362 (Zolbetuximab [previously named Claudiximab]) which is directed against CLDN18.2 has been developed by Ganymed Pharmaceuticals AG. This antibody comprises a heavy chain having the sequence set forth in SEQ ID NO: 51 and a light chain having the sequence set forth in SEQ ID NO: 24. IMAB362 recognizes the first extracellular domain (ECD1) of CLDN18.2 with high affinity and specificity. IMAB362 does not bind to any other claudin family member including the closely related splice variant 1 of Claudin 18 (CLDN18.1). IMAB362 shows precise tumor cell specificity and bundles two independent highly potent mechanisms of action. Upon target binding, IMAB362 mediates cell killing mainly by ADCC and CDC. Thus, IMAB362 lyses efficiently CLDN18.2-positive cells, including human gastric cancer cell lines in vitro and in vivo. Anti-tumor efficacy of IMAB362 was demonstrated in mice carrying xenografted tumors inoculated with CLDN18.2-positive cancer cell lines. Furthermore, IMAB362 has been evaluated in clinical studies as a single agent and in combination with epirubicin, oxaliplatin and capecitabine (EOX) chemotherapy or in combination with immunomodulation therapy (zoledronic acid [ZA] with or without interleukin-2 [IL-2]) for the treatment of adult subjects with CLDN18.2-positive advanced adenocarcinoma of the stomach, esophagus or GEJ. The poor prognosis of certain cancers such as, e.g., gastroesophageal cancers, highlights the need for additional treatment approaches.

Here we describe that combined administration of an anti-CLDN18.2 antibody, a platinum compound, a fluoropyrimidine compound or precursor thereof and an immune checkpoint inhibitor selected from a PD-1 inhibitor and a PD-L1 inhibitor is effective in treating CLDN18.2-expressing cancers such as CLDN18.2-positive adenocarcinomas of the stomach and eso-gastric junction.

### SUMMARY OF THE INVENTION

The present invention generally provides a combination therapy for effectively treating and/or preventing diseases associated with cells expressing CLDN18.2, including cancer diseases such as gastric cancer, esophageal cancer, pancreatic cancer, lung cancer such as non small cell lung cancer (NSCLC), ovarian cancer, colon cancer, hepatic cancer, head-neck cancer, and cancer of the gallbladder and metastases thereof, in particular gastric cancer metastasis such as Krukenberg tumors, peritoneal metastasis, liver metastasis and lymph node metastasis. Particularly preferred cancer diseases are adenocarcinomas of the stomach, the esophagus, the pancreatic duct, the bile ducts, the lung and the ovary.

In one aspect, the present invention provides a method for treating a patient, comprising administering to the patient an anti-CLDN18.2 antibody, a platinum compound, a fluoropyrimidine compound or precursor thereof and an immune checkpoint inhibitor selected from a PD-1 inhibitor and a PD-L1 inhibitor.

In one aspect, the present invention provides a method for treating or preventing cancer in a patient, comprising administering to the patient an anti-CLDN18.2 antibody, a platinum compound, a fluoropyrimidine compound or precursor thereof and an immune checkpoint inhibitor selected from a PD-1 inhibitor and a PD-L1 inhibitor.

In a further aspect, the present invention provides a method for inhibiting growth of a tumor in a patient having cancer, comprising administering to the patient an anti-CLDN18.2 antibody, a platinum compound, a fluoropyrimidine compound or precursor thereof and an immune checkpoint inhibitor selected from a PD-1 inhibitor and a PD-L1 inhibitor.

In one embodiment of all aspects disclosed herein, the platinum compound is oxaliplatin.

In one embodiment of all aspects disclosed herein, the fluoropyrimidine compound or precursor thereof is selected from the group consisting of fluorouracil (5-FU), capecitabine, floxuridine, tegafur, doxifluridine, and carmofur. In one embodiment of all aspects disclosed herein, the fluoropyrimidine compound or precursor thereof is fluorouracil (5-FU) or capecitabine. In one embodiment of all aspects disclosed herein, the fluoropyrimidine compound or precursor thereof is fluorouracil (5-FU).

In one embodiment of all aspects disclosed herein, the method comprises administration of oxaliplatin and 5-fluorouracil or a precursor thereof.

In one embodiment of all aspects disclosed herein, the method comprises administration of oxaliplatin and 5-fluorouracil or oxaliplatin and capecitabine.

In one embodiment of all aspects disclosed herein, the method comprises administration of folinic acid.

In one embodiment of all aspects disclosed herein, the method comprises administration of a mFOLFOX6 chemotherapy regimen.

In one embodiment of all aspects disclosed herein, the immune checkpoint inhibitor is selected from an anti-PD-1 antibody and an anti-PD-L1 antibody.

In one embodiment of all aspects disclosed herein, the immune checkpoint inhibitor is an anti-PD-1 antibody. In one embodiment of all aspects disclosed herein, the anti-PD-1 antibody is nivolumab (OPDIVO; BMS-936558), pembrolizumab (KEYTRUDA; MK-3475), pidilizumab (CT-011), cemiplimab (LIBTAYO, REGN2810), spartalizumab (PDR001), MEDI0680 (AMP-514), dostarlimab (TSR-042), cetrelimab (JNJ 63723283), toripalimab (JS001), AMP-224 (GSK-2661380), PF-06801591, tislelizumab (BGB-A317), ABBV-181, BI 754091, or SHR-1210.

In one embodiment of all aspects disclosed herein, the immune checkpoint inhibitor is nivolumab.

In one embodiment of all aspects disclosed herein, the immune checkpoint inhibitor is an anti-PD-L1 antibody. In one embodiment of all aspects disclosed herein, the anti-PD-L1 antibody is atezolizumab (TECENTRIQ; RG7446; MPDL3280A; R05541267), durvalumab (MEDI4736), BMS-936559, avelumab (bavencio), lodapolimab (LY3300054), CX-072 (Proclaim-CX-072), FAZ053, KN035, or MDX-1105.

In one embodiment of all aspects disclosed herein, the method in addition to administration of the anti-CLDN18.2 antibody comprises administration of oxaliplatin, 5-fluorouracil, folinic acid and nivolumab.

In one embodiment of all aspects disclosed herein, the method in addition to administration of the anti-CLDN18.2 antibody comprises administration of mFOLFOX6 chemotherapy regimen and nivolumab.

In one embodiment of all aspects disclosed herein, the anti-CLDN18.2 antibody binds to native epitopes of CLDN18.2 present on the surface of living cells. In one embodiment of all aspects disclosed herein, the anti-CLDN18.2 antibody is a monoclonal, chimeric or humanized antibody, or a fragment of an antibody. In one embodiment of all aspects disclosed herein, the anti-CLDN18.2 antibody is coupled to a therapeutic agent such as a toxin, a radioisotope, a drug or a cytotoxic agent.

In one embodiment of all aspects disclosed herein, the anti-CLDN18.2 antibody binds to the first extracellular loop of CLDN18.2.

In one embodiment of all aspects disclosed herein, the anti-CLDN18.2 antibody mediates cell killing by one or more of complement-dependent cytotoxicity (CDC) mediated lysis, antibody-dependent cell-mediated cytotoxicity (ADCC) mediated lysis, induction of apoptosis and inhibition of proliferation.

In one embodiment of all aspects disclosed herein, the anti-CLDN18.2 antibody is an antibody selected from the group consisting of:
(i) an antibody produced by and/or obtainable from a clone deposited under the accession no. DSM ACC2737, DSM ACC2738, DSM ACC2739, DSM ACC2740, DSM ACC2741, DSM ACC2742, DSM ACC2743, DSM ACC2745, DSM ACC2746, DSM ACC2747, DSM ACC2748, DSM ACC2808, DSM ACC2809, or DSM ACC2810,
(ii) an antibody which is a chimerized or humanized form of the antibody under (i),
(iii) an antibody having the specificity of the antibody under (i), and
(iv) an antibody comprising the antigen binding portion or antigen binding site, in particular the variable region, of the antibody under (i) and preferably having the specificity of the antibody under (i).

In one embodiment of all aspects disclosed herein, the anti-CLDN18.2 antibody comprises a heavy chain variable region CDR1 comprising the sequence of positions 45-52 of the sequence set forth in SEQ ID NO: 17, a heavy chain variable region CDR2 comprising the sequence of positions 70-77 of the sequence set forth in SEQ ID NO: 17, a heavy chain variable region CDR3 comprising the sequence of positions 116-126 of the sequence set forth in SEQ ID NO: 17, a light chain variable region CDR1 comprising the sequence of positions 47-58 of the sequence set forth in SEQ ID NO: 24, a light chain variable region CDR2 comprising the sequence of positions 76-78 of the sequence set forth in SEQ ID NO: 24, and a light chain variable region CDR3 comprising the sequence of positions 115-123 of the sequence set forth in SEQ ID NO: 24.

In one embodiment of all aspects disclosed herein, the anti-CLDN18.2 antibody comprises a heavy chain variable region comprising the sequence set forth in SEQ ID NO: 32 or a functional variant thereof, or a fragment of the amino acid sequence or functional variant and/or a light chain variable region comprising the sequence set forth in SEQ ID NO: 39 or a functional variant thereof, or a fragment of the amino acid sequence or functional variant.

In one embodiment of all aspects disclosed herein, the anti-CLDN18.2 antibody comprises a heavy chain constant region comprising the sequence set forth in SEQ ID NO: 13 or 52, or a functional variant thereof, or a fragment of the amino acid sequence or functional variant.

In one embodiment of all aspects disclosed herein, the anti-CLDN18.2 antibody comprises a heavy chain comprising the sequence set forth in SEQ ID NO: 17 or 51, or a functional variant thereof, or a fragment of the amino acid sequence or functional variant and/or a light chain comprising the sequence set forth in SEQ ID NO: 24 or a functional variant thereof, or a fragment of the amino acid sequence or functional variant.

In one embodiment of all aspects disclosed herein, the method comprises administering the anti-CLDN18.2 antibody at a dose of up to 1000 mg/m2. In one embodiment of all aspects disclosed herein, the method comprises administering the anti-CLDN18.2 antibody repeatedly at a dose of 300 to 600 mg/m2. In one embodiment of all aspects disclosed herein, the method comprises administering an initial dose of the anti-CLDN18.2 antibody of between 600 to 1000 mg/m2, such as 800 mg/m2, followed by administering the anti-CLDN18.2 antibody repeatedly at a dose of between 300 to 800 mg/m2, such as 400 mg/m2. In various embodiments, repeated administration involves administrations every 2 to 4 weeks such as every 2 weeks. In one embodiment of all aspects disclosed herein, the method comprises administering the anti-CLDN18.2 antibody according to one of the following possibilities:
(i) 800 mg/m2 initial dose followed by subsequent doses of 600 mg/m2 every 3 weeks;
(ii) 600 mg/m2 initial dose followed by subsequent doses of 600 mg/m2 every 3 weeks;
(iii) 800 mg/m2 initial dose followed by subsequent doses of 400 mg/m2 every 2 weeks; or
(iv) 600 mg/m2 initial dose followed by subsequent doses of 400 mg/m2 every 2 weeks.

In one embodiment of all aspects disclosed herein, the method comprises administering the anti-CLDN18.2 antibody as an intravenous (IV) infusion, e.g., as a minimum 2-hour intravenous (IV) infusion. IV infusion may be interrupted or slowed down to manage toxicity.

In one embodiment of all aspects disclosed herein, the cancer is CLDN18.2 positive. In one embodiment of all aspects disclosed herein, the cancer is selected from the group consisting of gastric cancer, esophageal cancer, pancreatic cancer, lung cancer, ovarian cancer, colon cancer, hepatic cancer, head-neck cancer, cancer of the gallbladder and the metastasis thereof. In one embodiment of all aspects disclosed herein, the cancer is a Krukenberg tumor, peritoneal metastasis, liver metastasis and/or lymph node metastasis. In one embodiment of all aspects disclosed herein, the cancer is an adenocarcinoma, in particular an advanced adenocarcinoma. In one embodiment of all aspects disclosed herein, the cancer is selected from the group consisting of cancer of the stomach, cancer of the esophagus, in particular the lower esophagus, cancer of the eso-gastric junction and gastroesophageal cancer.

In one embodiment of all aspects disclosed herein, the cancer is CLDN18.2-positive adenocarcinoma of the stomach and eso-gastric junction. In one embodiment of all aspects disclosed herein, the cancer is metastatic or locally advanced CLDN18.2-positive adenocarcinoma of the stomach and eso-gastric junction. In one embodiment of all aspects disclosed herein, the cancer is metastatic or locally advanced CLDN18.2-positive, HER2-negative adenocarcinoma of the stomach and eso-gastric junction.

In one embodiment of all aspects disclosed herein, the method comprises administration of an anti-CLDN18.2 antibody comprising a heavy chain comprising the sequence set forth in SEQ ID NO: 17 or 51, and a light chain comprising the sequence set forth in SEQ ID NO: 24, oxaliplatin, 5-fluorouracil, folinic acid and nivolumab.

In one embodiment of all aspects disclosed herein, CLDN18.2 has the amino acid sequence according to SEQ ID NO: 1.

In a further aspect, the present invention provides a medical preparation comprising an anti-CLDN18.2 antibody, a platinum compound, a fluoropyrimidine compound or precursor thereof and an immune checkpoint inhibitor selected from a PD-1 inhibitor and a PD-L1 inhibitor.

In one embodiment of all aspects disclosed herein, the medical preparation is a kit. In one embodiment, the kit comprises the anti-CLDN18.2 antibody, platinum compound, fluoropyrimidine compound or precursor thereof and immune checkpoint inhibitor selected from a PD-1 inhibitor and a PD-L1 inhibitor in separate containers.

In one embodiment of all aspects disclosed herein, the medical preparation further includes printed instructions for use of the preparation for treatment of cancer, in particular for use of the preparation in a method of the invention. Different embodiments of the medical preparation, and, in particular, of the anti-CLDN18.2 antibody, platinum compound, fluoropyrimidine compound or precursor thereof and immune checkpoint inhibitor selected from a PD-1 inhibitor and a PD-L1 inhibitor are as described above for the method of the invention.

The present invention also provides the agents described herein such as the anti-CLDN18.2 antibody, platinum compound, fluoropyrimidine compound or precursor thereof and immune checkpoint inhibitor selected from a PD-1 inhibitor and a PD-L1 inhibitor for use in therapy. In one embodiment, such therapy comprises treating and/or preventing diseases associated with cells expressing CLDN18.2, including cancer diseases such as those described herein.

The present invention also provides one or more of the agents described herein such as the anti-CLDN18.2 antibody for use in the methods described herein, e.g., the anti-CLDN18.2 antibody for administration in combination with a platinum compound, a fluoropyrimidine compound or precursor thereof and an immune checkpoint inhibitor selected from a PD-1 inhibitor and a PD-L1 inhibitor. The present invention also provides a use of one or more of the agents described herein such as the anti-CLDN18.2 antibody for the preparation of a pharmaceutical composition for use in the methods described herein, e.g., the use of the anti-CLDN18.2 antibody for the preparation of a pharmaceutical composition for administration in combination with a platinum compound, a fluoropyrimidine compound or precursor thereof and an immune checkpoint inhibitor selected from a PD-1 inhibitor and a PD-L1 inhibitor.

In one aspect, the present invention provides an anti-CLDN18.2 antibody for use in a method for treating or preventing cancer in a patient, comprising administering to the patient the anti-CLDN18.2 antibody, a platinum compound, a fluoropyrimidine compound or precursor thereof and an immune checkpoint inhibitor selected from a PD-1 inhibitor and a PD-L1 inhibitor. In a further aspect, the present invention provides an anti-CLDN18.2 antibody for use in a method for inhibiting growth of a tumor in a patient having cancer, comprising administering to the patient the anti-CLDN18.2 antibody, a platinum compound, a fluoropyrimidine compound or precursor thereof and an immune checkpoint inhibitor selected from a PD-1 inhibitor and a PD-L1 inhibitor. Preferred embodiments of these aspects are as described above for the methods of the invention.

In one aspect, the present invention provides an immune checkpoint inhibitor selected from a PD-1 inhibitor and a PD-L1 inhibitor for use in a method for treating or preventing cancer in a patient, comprising administering to the patient an anti-CLDN18.2 antibody, a platinum compound, a fluoropyrimidine compound or precursor thereof and the immune checkpoint inhibitor selected from a PD-1 inhibitor and a PD-L1 inhibitor. In a further aspect, the present invention provides an immune checkpoint inhibitor selected from a PD-1 inhibitor and a PD-L1 inhibitor for use in a method for inhibiting growth of a tumor in a patient having cancer, comprising administering to the patient an anti-CLDN18.2 antibody, a platinum compound, a fluoropyrimidine compound or precursor thereof and the immune checkpoint inhibitor selected from a PD-1 inhibitor and a PD-L1 inhibitor. Preferred embodiments of these aspects are as described above for the methods of the invention.

In one aspect, the present invention provides an anti-CLDN18.2 antibody, a platinum compound, a fluoropyrimidine compound or precursor thereof and an immune checkpoint inhibitor selected from a PD-1 inhibitor and a PD-L1 inhibitor for use in a method for treating or preventing cancer in a patient. In a further aspect, the present invention provides an anti-CLDN18.2 antibody, a platinum compound, a fluoropyrimidine compound or precursor thereof and an immune checkpoint inhibitor selected from a PD-1 inhibitor and a PD-L1 inhibitor for use in a method for inhibiting growth of a tumor in a patient having cancer. Preferred embodiments of these aspects are as described above for the methods of the invention.

In one aspect, the present invention provides a use of an anti-CLDN18.2 antibody for the preparation of a pharmaceutical composition for treating or preventing cancer in a patient, wherein the anti-CLDN18.2 antibody is to be administered together with a platinum compound, a fluoropyrimidine compound or precursor thereof and an immune checkpoint inhibitor selected from a PD-1 inhibitor and a PD-L1 inhibitor. In a further aspect, the present invention provides a use of an anti-CLDN18.2 antibody for the preparation of a pharmaceutical composition for inhibiting growth of a tumor in a patient having cancer, wherein the anti-CLDN18.2 antibody is to be administered together with a platinum compound, a fluoropyrimidine compound or precursor thereof and an immune checkpoint inhibitor selected from a PD-1 inhibitor and a PD-L1 inhibitor. Preferred embodiments of these aspects are as described above for the methods of the invention.

In one aspect, the present invention provides a use of an immune checkpoint inhibitor selected from a PD-1 inhibitor and a PD-L1 inhibitor for the preparation of a pharmaceutical composition for treating or preventing cancer in a patient, wherein the immune checkpoint inhibitor is to be administered together with an anti-CLDN18.2 antibody, a platinum compound, and a fluoropyrimidine compound or precursor thereof. In a further aspect, the present invention provides a use of an immune checkpoint inhibitor selected from a PD-1 inhibitor and a PD-L1 inhibitor for the preparation of a pharmaceutical composition for inhibiting growth of a tumor in a patient having cancer, wherein the immune checkpoint inhibitor is to be administered together with an anti-CLDN18.2 antibody, a platinum compound, and a fluoropyrimidine compound or precursor thereof. Preferred embodiments of these aspects are as described above for the methods of the invention.

In one aspect, the present invention provides a use of an anti-CLDN18.2 antibody, a platinum compound, a fluoropyrimidine compound or precursor thereof and an immune checkpoint inhibitor selected from a PD-1 inhibitor and a PD-L1 inhibitor for the preparation of a pharmaceutical composition for treating or preventing cancer in a patient. In a further aspect, the present invention provides a use of an anti-CLDN18.2 antibody, a platinum compound, a fluoropyrimidine compound or precursor thereof and an immune checkpoint inhibitor selected from a PD-1 inhibitor and a PD-L1 inhibitor for the preparation of a pharmaceutical composition for inhibiting growth of a tumor in a patient having cancer. Preferred embodiments of these aspects are as described above for the methods of the invention.

In one embodiment of the aspects described herein, a treatment described herein involves an immunotherapeutic treatment of a patient. In one embodiment of the aspects described herein, a treatment described herein involves inducing immune-mediated inhibition or destruction of cancer cells in a patient. In one embodiment of the aspects described herein, a treatment described herein involves inducing immune cell-mediated inhibition or destruction of cancer cells in a patient. In one embodiment of the aspects described herein, a treatment described herein involves inducing T cell-mediated inhibition or destruction of cancer cells in a patient. In one embodiment of the aspects described herein, a treatment described herein involves inducing NK cell-mediated inhibition or destruction of cancer cells in a patient. In one embodiment of the aspects described herein, a treatment described herein involves inducing antibody-dependent cell-mediated cytotoxicity (ADCC) against cancer cells in a patient. In one embodiment, ADCC is mediated, at least in part, by NK cells. In one embodiment of the aspects described herein, a treatment described herein involves inducing complement dependent cytotoxicity (CDC) against cancer cells in a patient.

In one embodiment of the aspects described herein, administration of the immune checkpoint inhibitor increases anti-tumor efficacy of the anti-CLDN18.2 antibody. In one embodiment of the aspects described herein, administration of the immune checkpoint inhibitor increases the efficacy of the anti-CLDN18.2 antibody to induce an immune-mediated inhibition or destruction of cancer cells in a patient. In one embodiment of the aspects described herein, administration of the immune checkpoint inhibitor increases the efficacy of the anti-CLDN18.2 antibody to induce an immune cell-mediated inhibition or destruction of cancer cells in a patient. In one embodiment of the aspects described herein, administration of the immune checkpoint inhibitor increases the efficacy of the anti-CLDN18.2 antibody to induce a T cell-mediated inhibition or destruction of cancer cells in a patient. In one embodiment of the aspects described herein, administration of the immune checkpoint inhibitor increases the efficacy of the anti-CLDN18.2 antibody to induce a NK cell-mediated inhibition or destruction of cancer cells in a patient. In one embodiment of the aspects described herein, administration of the immune checkpoint inhibitor increases the efficacy of the anti-CLDN18.2 antibody to induce antibody-dependent cell-mediated cytotoxicity (ADCC) against cancer cells in a patient. In one embodiment of the aspects described herein, administration of the immune checkpoint inhibitor increases the efficacy of the anti-CLDN18.2 antibody to induce complement dependent cytotoxicity (CDC) against cancer cells in a patient. In one embodiment of the aspects described herein, administration of the immune checkpoint inhibitor increases efficacy of the anti-CLDN18.2 antibody in a synergistic manner.

Other features and advantages of the instant invention will be apparent from the following detailed description and claims.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present invention will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e., the subject-matter consists in the inclusion of a stated member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The term “CLDN18” relates to claudin 18 and includes any variants, including claudin 18 splice variant 1 (claudin 18.1 (CLDN18.1)) and claudin 18 splice variant 2 (claudin 18.2 (CLDN18.2)).

The term “CLDN18.2” preferably relates to human CLDN18.2, and, in particular, to a protein comprising, preferably consisting of, the amino acid sequence according to SEQ ID NO: 1 of the sequence listing or a variant of said amino acid sequence.

The term “CLDN18.1” preferably relates to human CLDN18.1, and, in particular, to a protein comprising, preferably consisting of, the amino acid sequence according to SEQ ID NO: 2 of the sequence listing or a variant of said amino acid sequence.

The term “variant” according to the invention refers, in particular, to mutants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. A species homolog is a nucleic acid or amino acid sequence with a different species of origin from that of a given nucleic acid or amino acid sequence. The term “variant” shall encompass any posttranslationally modified variants and conformation variants.

According to the invention, the term “CLDN18.2 positive cancer” means a cancer involving cancer cells expressing CLDN18.2, preferably on the surface of said cancer cells.

“Cell surface” is used in accordance with its normal meaning in the art, and thus includes the outside of the cell which is accessible to binding by proteins and other molecules.

CLDN18.2 is expressed on the surface of cells if it is located at the surface of said cells and is accessible to binding by CLDN18.2-specific antibodies added to the cells.

According to the invention, CLDN18.2 is not substantially expressed in a cell if the level of expression is lower compared to expression in stomach cells or stomach tissue. Preferably, the level of expression is less than 10%, preferably less than 5%, 3%, 2%, 1%, 0.5%, 0.1% or 0.05% of the expression in stomach cells or stomach tissue or even lower. Preferably, CLDN18.2 is not substantially expressed in a cell if the level of expression exceeds the level of expression in non-cancerous tissue other than stomach by no more than 2-fold, preferably 1,5-fold, and preferably does not exceed the level of expression in said non-cancerous tissue. Preferably, CLDN18.2 is not substantially expressed in a cell if the level of expression is below the detection limit and/or if the level of expression is too low to allow binding by CLDN18.2-specific antibodies added to the cells.

According to the invention, CLDN18.2 is expressed in a cell if the level of expression exceeds the level of expression in non-cancerous tissue other than stomach preferably by more than 2-fold, preferably 10-fold, 100-fold, 1000-fold, or 10000-fold. Preferably, CLDN18.2 is expressed in a cell if the level of expression is above the detection limit and/or if the level of expression is high enough to allow binding by CLDN18.2-specific antibodies added to the cells. Preferably, CLDN18.2 expressed in a cell is expressed or exposed on the surface of said cell.

According to the invention, the term “disease” refers to any pathological state, including cancer, in particular those forms of cancer described herein. Any reference herein to cancer or particular forms of cancer also includes cancer metastasis thereof. In a preferred embodiment, a disease to be treated according to the present application involves cells expressing CLDN18.2.

“Diseases associated with cells expressing CLDN18.2” or similar expressions means according to the invention that CLDN18.2 is expressed in cells of a diseased tissue or organ. In one embodiment, expression of CLDN18.2 in cells of a diseased tissue or organ is increased compared to the state in a healthy tissue or organ. An increase refers to an increase by at least 10%, in particular at least 20%, at least 50%, at least 100%, at least 200%, at least 500%, at least 1000%, at least 10000% or even more. In one embodiment, expression is only found in a diseased tissue, while expression in a healthy tissue is repressed. According to the invention, diseases associated with cells expressing CLDN18.2 include cancer diseases. Furthermore, according to the invention, cancer diseases preferably are those wherein the cancer cells express CLDN18.2.

As used herein, a “cancer disease” or “cancer” includes a disease characterized by aberrantly regulated cellular growth, proliferation, differentiation, adhesion, and/or migration. By “cancer cell” is meant an abnormal cell that grows by a rapid, uncontrolled cellular proliferation and continues to grow after the stimuli that initiated the new growth cease. Preferably, a “cancer disease” is characterized by cells expressing CLDN18.2 and a cancer cell expresses CLDN18.2. A cell expressing CLDN18.2 preferably is a cancer cell, preferably of the cancers described herein.

“Adenocarcinoma” is a cancer that originates in glandular tissue. This tissue is also part of a larger tissue category known as epithelial tissue. Epithelial tissue includes skin, glands and a variety of other tissue that lines the cavities and organs of the body. Epithelium is derived embryologically from ectoderm, endoderm and mesoderm. To be classified as adenocarcinoma, the cells do not necessarily need to be part of a gland, as long as they have secretory properties. This form of carcinoma can occur in some higher mammals, including humans. Well differentiated adenocarcinomas tend to resemble the glandular tissue that they are derived from, while poorly differentiated may not. By staining the cells from a biopsy, a pathologist will determine whether the tumor is an adenocarcinoma or some other type of cancer. Adenocarcinomas can arise in many tissues of the body due to the ubiquitous nature of glands within the body. While each gland may not be secreting the same substance, as long as there is an exocrine function to the cell, it is considered glandular and its malignant form is therefore named adenocarcinoma. Malignant adenocarcinomas invade other tissues and often metastasize given enough time to do so. Ovarian adenocarcinoma is the most common type of ovarian carcinoma. It includes the serous and mucinous adenocarcinomas, the clear cell adenocarcinoma and the endometrioid adenocarcinoma.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor and the regional lymph node system. In one embodiment, the term “metastasis” according to the invention relates to lymph node metastasis. One particular form of metastasis which is treatable using the therapy of the invention is metastasis originating from gastric cancer as primary site. In preferred embodiments such gastric cancer metastasis is Krukenberg tumors, peritoneal metastasis, liver metastasis and/or lymph node metastasis.

Krukenberg tumor is an uncommon metastatic tumor of the ovary accounting for 1% to 2% of all ovarian tumors. Prognosis of Krukenberg tumor is still very poor and there is no established treatment for Krukenberg tumors. Krukenberg tumor is a metastatic signet ring cell adenocarcinoma of the ovary. Stomach is the primary site in most Krukenberg tumor cases (70%). Carcinomas of colon, appendix, and breast (mainly invasive lobular carcinoma) are the next most common primary sites. Rare cases of Krukenberg tumor originating from carcinomas of the gallbladder, biliary tract, pancreas, small intestine, ampulla of Vater, cervix, and urinary bladder/urachus have been reported.

The interval between the diagnosis of a primary carcinoma and the subsequent discovery of ovarian involvement is usually 6 months or less, but longer periods have been reported. In many cases, the primary tumor is very small and can escape detection. A history of a prior carcinoma of the stomach or another organ can be obtained in only 20% to 30% of the cases. Krukenberg tumor is an example of the selective spread of cancers, most commonly in the stomach-ovarian axis. This axis of tumor spread has historically drawn the attention of many pathologists, especially when it was found that gastric neoplasms selectively metastasize to the ovaries without involvement of other tissues. The route of metastasis of gastric carcinoma to the ovaries has been a mystery for a long time, but it is now evident that retrograde lymphatic spread is the most likely route of metastasis. Women with Krukenberg tumors tend to be unusually young for patients with metastatic carcinoma as they are typically in the fifth decade of their lives, with an average age of 45 years. This young age of distribution can be related in part to the increased frequency of gastric signet ring cell carcinomas in young women. Common presenting symptoms are usually related to ovarian involvement, the most common of which are abdominal pain and distension (mainly because of the usually bilateral and often large ovarian masses). The remaining patients have nonspecific gastrointestinal symptoms or are asymptomatic. In addition, Krukenberg tumor is reportedly associated with virilization resulting from hormone production by ovarian stroma. Ascites is present in 50% of the cases and usually reveals malignant cells. Krukenberg tumors are bilateral in more than 80% of the reported cases. The ovaries are usually asymmetrically enlarged, with a bosselated contour. The sectioned surfaces are yellow or white; they are usually solid, although they are occasionally cystic. Importantly, the capsular surface of the ovaries with Krukenberg tumors is typically smooth and free of adhesions or peritoneal deposits. Of note, other metastatic tumors to the ovary tend to be associated with surface implants. This may explain why the gross morphology of Krukenberg tumor can deceptively appear as a primary ovarian tumor. However, bilateralism in Krukenberg tumor is consistent with its metastatic nature. Patients with Krukenberg tumors have an overall mortality rate that is significantly high. Most patients die within 2 years (median survival, 14 months). Several studies show that the prognosis is poor when the primary tumor is identified after the metastasis to the ovary is discovered, and the prognosis becomes worse if the primary tumor remains covert. No optimal treatment strategy for Krukenberg tumors has been clearly established in the literature. Whether a surgical resection should be performed has not been adequately addressed. Chemotherapy or radiotherapy has no significant effect on prognosis of patients with Krukenberg tumors.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

As used herein, “immune checkpoint” refers to regulators of the immune system, and, in particular, co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1 and/or PD-L2.

The “Programmed Death-1 (PD-1)” receptor refers to an immuno-inhibitory receptor belonging to the CD28 family. PD-1 is expressed predominantly on previously activated T cells in vivo, and binds to two ligands, PD-L1 (also known as B7-H1 or CD274) and PD-L2 (also known as B7-DC or CD273). The term “PD-1” as used herein includes human PD-1 (hPD-1), variants, isoforms, and species homologs of hPD-1, and analogs having at least one common epitope with hPD-1. “Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The term “PD-L2” as used herein includes human PD-L2 (hPD-L2), variants, isoforms, and species homologs of hPD-L2, and analogs having at least one common epitope with hPD-L2. The ligands of PD-1 (PD-L1 and PD-L2) are expressed on the surface of antigen-presenting cells, such as dendritic cells or macrophages, and other immune cells. Binding of PD-1 to PD-L1 or PD-L2 results in downregulation of T cell activation. Cancer cells expressing PD-L1 and/or PD-L2 are able to switch off T cells expressing PD-1 which results in suppression of the anticancer immune response. The interaction between PD-1 and its ligands results in a decrease in tumor infiltrating lymphocytes, a decrease in T cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

Many of the immune checkpoints are regulated by interactions between specific receptor and ligand pairs, such as those described above. Thus, immune checkpoint proteins mediate immune checkpoint signaling. For example, checkpoint proteins directly or indirectly regulate T cell activation, T cell proliferation and/or T cell function. Cancer cells often exploit these checkpoint pathways to protect themselves from being attacked by the immune system. Hence, the function of checkpoint proteins, which is modulated according to the present disclosure is typically the regulation of T cell activation, T cell proliferation and/or T cell function. Immune checkpoint proteins thus regulate and maintain self-tolerance and the duration and amplitude of physiological immune responses.

As used herein, the term “immune checkpoint modulator” or “checkpoint modulator” refers to a molecule or to a compound that modulates the function of one or more checkpoint proteins. Immune checkpoint modulators are typically able to modulate self-tolerance and/or the amplitude and/or the duration of the immune response. Preferably, the immune checkpoint modulator used according to the present disclosure modulates the function of one or more human checkpoint proteins and is, thus, a “human checkpoint modulator”. Specifically, the human checkpoint modulator as used herein is an immune checkpoint inhibitor.

As used herein, “immune checkpoint inhibitor” or “checkpoint inhibitor” refers to a molecule that totally or partially reduces, inhibits, interferes with or negatively modulates one or more checkpoint proteins or that totally or partially reduces, inhibits, interferes with or negatively modulates expression of one or more checkpoint proteins. In certain embodiments, the immune checkpoint inhibitor binds to one or more checkpoint proteins. In certain embodiments, the immune checkpoint inhibitor binds to one or more molecules regulating checkpoint proteins. In certain embodiments, the immune checkpoint inhibitor binds to precursors of one or more checkpoint proteins e.g., on DNA- or RNA-level. Any agent that functions as a checkpoint inhibitor according to the present disclosure can be used.

The term “partially” as used herein means at least 5%, 10%, 15%, 20%, 25%, 30%, 35%, 40%, 45%, 50%, 55%, 60%, 65%, 70%, 75%, 80%, 85%, 90%, 95%, 96%, 97%, 98% or 99% in the level, e.g., in the level of inhibition of a checkpoint protein.

In certain embodiments, the immune checkpoint inhibitor suitable for use herein is an antagonist of inhibitory signals, e.g., an antibody which targets, for example, PD-1, or PD-L1.

In certain embodiments, the immune checkpoint inhibitor prevents inhibitory signals associated with the immune checkpoint. In certain embodiments, the immune checkpoint inhibitor is an antibody, or fragment thereof that disrupts inhibitory signaling associated with the immune checkpoint. In certain embodiments, the immune checkpoint inhibitor is a small molecule inhibitor that disrupts inhibitory signaling. In certain embodiments, the immune checkpoint inhibitor is a peptide-based inhibitor that disrupts inhibitory signaling. In certain embodiments, the immune checkpoint inhibitor is an inhibitory nucleic acid molecule that disrupts inhibitory signaling.

In certain embodiments, the immune checkpoint inhibitor is an antibody, fragment thereof, or antibody mimic, that prevents the interaction between checkpoint blocker proteins, e.g., an antibody, or fragment thereof that prevents the interaction between PD-1 and PD-L1 or PD-L2.

Inhibiting or blocking of inhibitory immune checkpoint signaling, as described herein, results in preventing or reversing immune-suppression and establishment or enhancement of T cell immunity against cancer cells. In one embodiment, inhibition of immune checkpoint signaling, as described herein, reduces or inhibits dysfunction of the immune system. In one embodiment, inhibition of immune checkpoint signaling, as described herein, renders dysfunctional immune cells less dysfunctional. In one embodiment, inhibition of immune checkpoint signaling, as described herein, renders a dysfunctional T cell less dysfunctional.

The term “dysfunction”, as used herein, refers to a state of reduced immune responsiveness to antigenic stimulation. The term includes the common elements of both exhaustion and/or anergy in which antigen recognition may occur, but the ensuing immune response is ineffective to control infection or tumor growth. Dysfunction also includes a state in which antigen recognition is retarded due to dysfunctional immune cells.

The term “dysfunctional”, as used herein, also refers to an immune cell that is in a state of reduced immune responsiveness to antigen stimulation. Dysfunctional includes unresponsive to antigen recognition and impaired capacity to translate antigen recognition into downstream T cell effector functions, such as proliferation, cytokine production (e.g., IL-2) and/or target cell killing.

The term “anergy”, as used herein, refers to the state of unresponsiveness to antigen stimulation resulting from incomplete or insufficient signals delivered through the T cell receptor (TCR). T cell anergy can also result upon stimulation with antigen in the absence of co-stimulation, resulting in the cell becoming refractory to subsequent activation by the antigen even in the context of co-stimulation. The unresponsive state can often be overridden by the presence of IL-2. Anergic T cells do not undergo clonal expansion and/or acquire effector functions.

The term “exhaustion”, as used herein, refers to immune cell exhaustion, such as T cell exhaustion as a state of T cell dysfunction that arises from sustained TCR signaling that occurs during many chronic infections and cancer. It is distinguished from anergy in that it arises not through incomplete or deficient signaling, but from sustained signaling. Exhaustion is defined by poor effector function, sustained expression of inhibitory receptors and a transcriptional state distinct from that of functional effector or memory T cells. Exhaustion prevents optimal control of diseases (e.g., infection and tumors). Exhaustion can result from both extrinsic negative regulatory pathways (e.g., immunoregulatory cytokines) as well as cell intrinsic negative regulatory pathways (inhibitory immune checkpoint pathways, such as described herein).

“Enhancing T cell function” means to induce, cause or stimulate a T cell to have a sustained or amplified biological function, or renew or reactivate exhausted or inactive T cells. Examples of enhancing T cell function include increased secretion of Y-interferon from CD8+ T cells, increased proliferation, increased antigen responsiveness (e.g., tumor clearance) relative to such levels before the intervention. In one embodiment, the level of enhancement is as least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 200%, or more. Manners of measuring this enhancement are known to one of ordinary skill in the art.

The immune checkpoint inhibitor may be an inhibitory nucleic acid molecule. The term “inhibitory nucleic acid” or “inhibitory nucleic acid molecule” as used herein refers to a nucleic acid molecule, e.g., DNA or RNA, that totally or partially reduces, inhibits, interferes with or negatively modulates one or more checkpoint proteins. Inhibitory nucleic acid molecules include, without limitation, oligonucleotides, siRNA, shRNA, antisense DNA or RNA molecules, and aptamers (e.g., DNA or RNA aptamers).

The term “oligonucleotide” as used herein refers to a nucleic acid molecule that is able to decrease protein expression, in particular expression of a checkpoint protein, such as the checkpoint proteins described herein. Oligonucleotides are short DNA or RNA molecules, typically comprising from 2 to 50 nucleotides. Oligonucleotides maybe single-stranded or double-stranded. A checkpoint inhibitor oligonucleotide may be an antisense-oligonucleotide. Antisense-oligonucleotides are single-stranded DNA or RNA molecules that are complementary to a given sequence, in particular to a sequence of the nucleic acid sequence (or a fragment thereof) of a checkpoint protein. Antisense RNA is typically used to prevent protein translation of mRNA, e.g., of mRNA encoding a checkpoint protein, by binding to said mRNA. Antisense DNA is typically used to target a specific, complementary (coding or non-coding) RNA. If binding takes place, such a DNA/RNA hybrid can be degraded by the enzyme RNase H. Moreover, morpholino antisense oligonucleotides can be used for gene knockdowns in vertebrates. For example, Kryczek et al., 2006 (J Exp Med, 203:871-81) designed B7-H4-specific morpholinos that specifically blocked B7-H4 expression in macrophages, resulting in increased T cell proliferation and reduced tumor volumes in mice with tumor associated antigen (TAA)-specific T cells.

The terms “siRNA” or “small interfering RNA” or “small inhibitory RNA” are used interchangeably herein and refer to a double-stranded RNA molecule with a typical length of 20-25 base pairs that interferes with expression of a specific gene, such as a gene coding for a checkpoint protein, with a complementary nucleotide sequence. In one embodiment, siRNA interferes with mRNA therefore blocking translation, e.g., translation of an immune checkpoint protein. Transfection of exogenous siRNA may be used for gene knockdown, however, the effect may be only transient, especially in rapidly dividing cells. Stable transfection may be achieved, e.g., by RNA modification or by using an expression vector. Useful modifications and vectors for stable transfection of cells with siRNA are known in the art. siRNA sequences may also be modified to introduce a short loop between the two strands resulting in a “small hairpin RNA” or “shRNA”. shRNA can be processed into a functional siRNA by Dicer. shRNA has a relatively low rate of degradation and turnover. Accordingly, the immune checkpoint inhibitor may be a shRNA.

The term “aptamer” as used herein refers to a single-stranded nucleic acid molecule, such as DNA or RNA, typically in a length of 25-70 nucleotides that is capable of binding to a target molecule, such as a polypeptide. In one embodiment, the aptamer binds to an immune checkpoint protein such as the immune checkpoint proteins described herein. For example, an aptamer according to the disclosure can specifically bind to an immune checkpoint protein or polypeptide, or to a molecule in a signaling pathway that modulates the expression of an immune checkpoint protein or polypeptide. The generation and therapeutic use of aptamers is well known in the art (see, e.g., U.S. Pat. No. 5,475,096).

The terms “small molecule inhibitor” or “small molecule” are used interchangeably herein and refer to a low molecular weight organic compound, usually up to 1000 daltons, that totally or partially reduces, inhibits, interferes with, or negatively modulates one or more checkpoint proteins as described above. Such small molecular inhibitors are usually synthesized by organic chemistry, but may also be isolated from natural sources, such as plants, fungi, and microbes. The small molecular weight allows a small molecule inhibitor to rapidly diffuse across cell membranes. For example, various A2AR antagonists known in the art are organic compounds having a molecular weight below 500 daltons.

The immune checkpoint inhibitor may be an antibody, an antigen-binding fragment thereof, an antibody mimic or a fusion protein comprising an antibody portion with an antigen-binding fragment of the required specificity. Antibodies or antigen-binding fragments thereof are as described herein. Antibodies or antigen-binding fragments thereof that are immune checkpoint inhibitors include in particular antibodies or antigen-binding fragments thereof that bind to immune checkpoint proteins, such as immune checkpoint receptors or immune checkpoint receptor ligands. Antibodies or antigen-binding fragments may also be conjugated to further moieties, as described herein. In particular, antibodies or antigen-binding fragments thereof are chimerized, humanized or human antibodies. Preferably, immune checkpoint inhibitor antibodies or antigen-binding fragments thereof are antagonists of immune checkpoint receptors or of immune checkpoint receptor ligands.

In a preferred embodiment, an antibody that is an immune checkpoint inhibitor, is an isolated antibody.

The antibody that is an immune checkpoint inhibitor or the antigen-binding fragment thereof according to the present disclosure may also be an antibody that cross-competes for antigen binding with any known immune checkpoint inhibitor antibody. In certain embodiments, an immune checkpoint inhibitor antibody cross-competes with one or more of the immune checkpoint inhibitor antibodies described herein. The ability of antibodies to cross-compete for binding to an antigen indicates that these antibodies may bind to the same epitope region of the antigen or when binding to another epitope sterically hinder the binding of known immune checkpoint inhibitor antibodies to that particular epitope region. These cross-competing antibodies may have functional properties very similar to those they are cross-competing with as they are expected to block binding of the immune checkpoint to its ligand either by binding to the same epitope or by sterically hindering the binding of the ligand. Cross-competing antibodies can be readily identified based on their ability to cross-compete with one or more of known antibodies in standard binding assays such as Surface Plasmon Resonance analysis, ELISA assays or flow cytometry (see, e.g., WO 2013/173223).

In certain embodiments, antibodies or antigen binding fragments thereof that cross-compete for binding to a given antigen with or bind to the same epitope region of a given antigen as, one or more known antibodies are monoclonal antibodies. For administration to human patients, these cross-competing antibodies can be chimeric antibodies, or humanized or human antibodies. Such chimeric, humanized or human monoclonal antibodies can be prepared and isolated by methods well known in the art.

The checkpoint inhibitor may also be in the form of the soluble form of the molecules (or variants thereof) themselves, e.g., a soluble PD-L1 or PD-L1 fusion.

In certain embodiments, the inhibitory immunoregulator (immune checkpoint blocker) is a component of the PD-1/PD-L1 or PD-1/PD-L2 signaling pathway. Accordingly, embodiments of the disclosure provide for administering to a subject a checkpoint inhibitor of the PD-1 signaling pathway. In certain embodiments, the checkpoint inhibitor of the PD-1 signaling pathway is a PD-1 inhibitor. In certain embodiments, the checkpoint inhibitor of the PD-1 signaling pathway is a PD-1 ligand inhibitor, such as a PD-L1 inhibitor or a PD-L2 inhibitor. In a preferred embodiment, the checkpoint inhibitor of the PD-1 signaling pathway is an antibody or an antigen-binding portion thereof that disrupts the interaction between the PD-1 receptor and one or more of its ligands, PD-L1 and/or PD-L2. Antibodies which bind to PD-1 and disrupt the interaction between PD-1 and one or more of its ligands are known in the art. In certain embodiments, the antibody or antigen-binding portion thereof binds specifically to PD-1. In certain embodiments, the antibody or antigen-binding portion thereof binds specifically to PD-L1 and inhibits its interaction with PD-1, thereby increasing immune activity. In certain embodiments, the antibody or antigen-binding portion thereof binds specifically to PD-L2 and inhibits its interaction with PD-1, thereby increasing immune activity.

Exemplary PD-1 inhibitors include, without limitation, anti-PD-1 antibodies such as BGB-A317 (BeiGene; see U.S. Pat. No. 8,735,553, WO 2015/35606 and US 2015/0079109), cemiplimab (Regeneron; see WO 2015/112800) and lambrolizumab (e.g., disclosed as hPD109A and its humanized derivatives h409A1, h409A16 and h409A17 in WO2008/156712), AB137132 (Abcam), EH12.2H7 and RMP1-14 (#BE0146; Bioxcell Lifesciences Pvt. LTD.), MIH4 (Affymetrix eBioscience), nivolumab (OPDIVO, BMS-936558; Bristol Myers Squibb; see WO 2006/121168), pembrolizumab (KEYTRUDA; MK-3475; Merck; see WO 2008/156712), pidilizumab (CT-011; CureTech; see Hardy et al., 1994, Cancer Res., 54(22):5793-6 and WO 2009/101611), PDR001 (Novartis; see WO 2015/112900), MEDI0680 (AMP-514; AstraZeneca; see WO 2012/145493), TSR-042 (see WO 2014/179664), REGN-2810 (H4H7798N; cf. US 2015/0203579), JS001 (TAIZHOU JUNSHI PHARMA; see Si-Yang Liu et al., 2007, J. Hematol. Oncol. 70: 136), AMP-224 (GSK-2661380; cf. Li et al., 2016, Int J Mol Sci 17(7):1151 and WO 2010/027827 and WO 2011/066342), PF-06801591 (Pfizer), BGB-A317 (BeiGene; see WO 2015/35606 and US 2015/0079109), BI 754091, SHR-1210 (see WO2015/085847), and antibodies 17D8, 2D3, 4H1, 4A11, 7D3, and 5F4 as described in WO 2006/121168, INCSHR1210 (Jiangsu Hengrui Medicine; also known as SHR-1210; see WO 2015/085847), TSR-042 (Tesaro Biopharmaceutical; also known as ANB011; see WO2014/179664), GLS-010 (Wuxi/Harbin Gloria Pharmaceuticals; also known as WBP3055; see Si-Yang et al., 2017, J. Hematol. Oncol. 70: 136), STI-1110 (Sorrento Therapeutics; see WO 2014/194302), AGEN2034 (Agenus; see WO 2017/040790), MGA012 (Macrogenics; see WO 2017/19846), IBI308 (Innovent; see WO 2017/024465, WO 2017/025016, WO 2017/132825, and WO 2017/133540), anti-PD-1 antibodies as described, e.g., in U.S. Pat. Nos. 7,488,802, 8,008,449, 8,168,757, WO 03/042402, WO 2010/089411 (further disclosing anti-PD-L1 antibodies), WO 2010/036959, WO 2011/159877 (further disclosing antibodies against TIM-3), WO 2011/082400, WO 2011/161699, WO 2009/014708, WO 03/099196, WO 2009/114335, WO 2012/145493 (further disclosing antibodies against PD-L1), WO 2015/035606, WO 2014/055648 (further disclosing anti-KIR antibodies), US 2018/0185482 (further disclosing anti-PD-L1 and anti-TIGIT antibodies), U.S. Pat. Nos. 8,008,449, 8,779,105, 6,808,710, 8,168,757, US 2016/0272708, and U.S. Pat. No. 8,354,509, small molecule antagonists to the PD-1 signaling pathway as disclosed, e.g., in Shaabani et al., 2018, Expert Op Ther Pat., 28(9):665-678 and Sasikumar and Ramachandra, 2018, BioDrugs, 32(5):481-497, siRNAs directed to PD-1 as disclosed, e.g., in WO 2019/000146 and WO 2018/103501, soluble PD-1 proteins as disclosed in WO 2018/222711 and oncolytic viruses comprising a soluble form of PD-1 as described, e.g., in WO 2018/022831.

In a certain embodiment, the PD-1 inhibitor is nivolumab (OPDIVO; BMS-936558), pembrolizumab (KEYTRUDA; MK-3475), pidilizumab (CT-011), PDR001, MEDI0680 (AMP-514), TSR-042, REGN2810, JS001, AMP-224 (GSK-2661380), PF-06801591, BGB-A317, BI 754091, or SHR-1210.

In some embodiments, the anti-PD-1 antibody is pembrolizumab (CAS Registry Number: 1374853-91-4). Pembrolizumab (Merck), also known as MK-3475, Merck 3475, lambrolizumab, KEYTRUDA®, and SCH-900475, is an anti-PD-1 antibody described in WO2009/114335. In some embodiments, the anti-PD-1 antibody comprises a heavy chain and a light chain sequence, wherein:

(a)
the heavy chain comprises the
amino acid sequence:
(SEQ ID NO: 53)
QVQLVQSGVE VKKPGASVKV SCKASGYTFT
NYYMYWVRQA PGQGLEWMGG INPSNGGTNE
NEKFKNRVTL TTDSSTTTAY MELKSLQFDD
TAVYYCARRD YRFDMGFDYW GQGTTVTVSS
ASTKGPSVFP LAPCSRSTSE STAALGCLVK
DYFPEPVTVS WNSGALTSGV HTFPAVLQSS
GLYSLSSVVT VPSSSLGTKT YTCNVDHKPS
NTKVDKRVES KYGPPCPPCP APEFLGGPSV
FLFPPKPKDT LMISRTPEVT CVVVDVSQED
PEVQFNWYVD GVEVHNAKTK PREEQFNSTY
RVVSVLTVLH QDWLNGKEYK CKVSNKGLPS
SIEKTISKAK GQPREPQVYT LPPSQEEMTK
NQVSLTCLVK GFYPSDIAVE WESNGQPENN
YKTTPPVLDS DGSFFLYSRL TVDKSRWQEG
NVFSCSVMHE ALHNHYTQKS LSLSLGK,
and
(b) the light chain comprises
the amino acid sequence:
(SEQ ID NO: 54)
EIVLTQSPAT LSLSPGERAT LSCRASKGVS
TSGYSYLHWY QQKPGQAPRL LIYLASYLES
GVPARESGSG SGTDETLTIS SLEPEDFAVY
YCQHSRDLPL TFGGGTKVEI KRTVAAPSVE
IFPPSDEQLK SGTASVVCLL NNFYPREAKV
QWKVDNALQS GNSQESVTEQ DSKDSTYSLS
STLTLSKADY EKHKVYACEV THQGLSSPVT
KSENRGEC.

In some embodiments, the anti-PD-1 antibody comprises the six CDR sequences from SEQ ID NO:53 and SEQ ID NO:54 (e.g., the three heavy chain CDRs from SEQ ID NO:53 and the three light chain CDRs from SEQ ID NO:54). In some embodiments, the anti-PD-1 antibody comprises the heavy chain variable domain from SEQ ID NO:53 and the light chain variable domain from SEQ ID NO:54. In some embodiments, the anti-PD-1 antibody comprises: a heavy chain variable region (VH) comprising an amino acid sequence of SEQ ID NO:55, and (b) a light chain variable region (VL) comprising an amino acid sequence of SEQ ID NO:56.

In some embodiments, the anti-PD-1 antibody comprises: (a) a heavy chain variable region (VH) that comprises a CDR-1 comprising an amino acid sequence of GYTFTNYY (SEQ ID NO:57), a CDR-2 comprising an amino acid sequence of INPSNGGT (SEQ ID NO:58), and a CDR-3 comprising an amino acid ARRDYRFDMGFDY (SEQ ID NO:59), and (b) a light chain variable region (VL) that comprises a CDR-1 comprising an amino acid sequence of KGVSTSGYSY (SEQ ID NO:60), a CDR-2 comprising an amino acid sequence of LAS (SEQ ID NO:61), and a CDR-3 comprising an amino acid sequence of QHSRDLPLT (SEQ ID NO:62).

In a certain embodiment, the anti-PD-1 antibody is pembrolizumab which may be administered at a dose of 200 mg intravenously. Pembrolizumab may be given intravenously according to institutional guidelines, published guidelines and the respective product prescribing information, and dosed according to this protocol.

In some embodiments, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO2006/121168. In some embodiments, the anti-PD-1 antibody comprises a heavy chain and a light chain sequence, wherein:

(a)
the heavy chain comprises the
amino acid sequence:
(SEQ ID NO: 63)
QVQLVESGGG VVQPGRSLRL DCKASGITES
NSGMHWVRQA PGKGLEWVAV IWYDGSKRYY
ADSVKGRFTI SRDNSKNTLF LQMNSLRAED
TAVYYCATND DYWGQGTLVT VSSASTKGPS
VFPLAPCSRS TSESTAALGC LVKDYFPEPV
TVSWNSGALT SGVHTFPAVL QSSGLYSLSS
VVTVPSSSLG TKTYTCNVDH KPSNTKVDKR
VESKYGPPCP PCPAPEFLGG PSVFLFPPKP
KDTLMISRTP EVTCVVVDVS QEDPEVQENW
YVDGVEVHNA KTKPREEQEN STYRVVSVLT
VLHQDWLNGK EYKCKVSNKG LPSSIEKTIS
KAKGQPREPQ VYTLPPSQEE MTKNQVSLTC
LVKGFYPSDI AVEWESNGQP ENNYKTTPPV
LDSDGSFFLY SRLTVDKSRW QEGNVFSCSV
MHEALHNHYT QKSLSLSLGK,
and
(b) the light chain comprises
the amino acid sequence:
(SEQ ID NO: 64)
EIVLTQSPAT LSLSPGERAT LSCRASQSVS
SYLAWYQQKP GQAPRLLIYD ASNRATGIPA
RFSGSGSGTD FTLTISSLEP EDFAVYYCQQ
SSNWPRTFGQ GTKVEIKRTV AAPSVFIFPP
SDEQLKSGTA SVVCLLNNFY PREAKVQWKV
DNALQSGNSQ ESVTEQDSKD STYSLSSTLT
LSKADYEKHK VYACEVTHQG LSSPVTKSEN
RGEC.

In some embodiments, the anti-PD-1 antibody comprises the six CDR sequences from SEQ ID NO:63 and SEQ ID NO:64 (e.g., the three heavy chain CDRs from SEQ ID NO:63 and the three light chain CDRs from SEQ ID NO:64). In some embodiments, the anti-PD-1 antibody comprises the heavy chain variable domain from SEQ ID NO:63 and the light chain variable domain from SEQ ID NO:64. In some embodiments, the anti-PD-1 antibody comprises: a heavy chain variable region (VH) comprising an amino acid sequence of SEQ ID NO:65, and (b) a light chain variable region (VL) comprising an amino acid sequence of SEQ ID NO:66.

In some embodiments, the anti-PD-1 antibody comprises: (a) a heavy chain variable region (VH) that comprises a CDR-1 comprising an amino acid sequence of GITFSNSG (SEQ ID NO:67), a CDR-2 comprising an amino acid sequence of IWYDGSKR (SEQ ID NO:68), and a CDR-3 comprising an amino acid ATNDDY (SEQ ID NO:69), and (b) a light chain variable region (VL) that comprises a CDR-1 comprising an amino acid sequence of QSVSSY (SEQ ID NO:70), a CDR-2 comprising an amino acid sequence of DAS (SEQ ID NO:71), and a CDR-3 comprising an amino acid sequence of QQSSNWPRT (SEQ ID NO:72).

In a certain embodiment, the anti-PD-1 antibody is nivolumab which may be administered at a dose of 240 mg intravenously. Nivolumab may be given intravenously according to institutional guidelines, published guidelines and the respective product prescribing information, and dosed according to this protocol.

Programmed death ligand 1 (PD-L1) is a protein that interacts with programmed death protein 1 (PD-1) and is expressed on, for example, immune and tumor cells. In certain embodiments, the subject for treatment has a cancer characterized by expression of PD-L1. In certain embodiments, a sample from a subject having cancer is characterized by expression of PD-L1. In certain embodiments, PD-L1 expression is determined using Immunohistochemistry (IHC).

In certain embodiments, PD-L1 expression is expressed as a Combined Positive Score (CPS). The Combined Positive Score (CPS) of the sample can be determined by dividing the number of PD-L1 stained cells (tumor cells, lymphocytes and macrophages) by the total number of viable tumor cells, and then multiplying by 100. In certain embodiments, the Combined Positive Score (CPS) refers to the ratio of the number of PD-L1 positive tumor cells and PD-L1 positive mononuclear inflammatory cells (MIC) within the tumor nests and the adjacent supporting stroma (numerator) divided by the total number of tumor cells (denominator; i.e., the number of PD-L1 positive and PD-L1 negative tumor cells), and then multiplying by 100. PD-L1 expression at any intensity may be considered positive, i.e., weak (1+), moderate (2+), or strong (3+).

In certain embodiments, a sample expressing PD-L1 has a CPS of at least about 1 (i.e., CPS≥1). In certain embodiments, a sample expressing PD-L1 has a CPS of at least about 5 (i.e., CPS≥5). In certain embodiments, a sample expressing PD-L1 has a CPS of at least about 10 (i.e., CPS.≥10).

Exemplary PD-1 ligand inhibitors are PD-L1 inhibitors and PD-L2 inhibitors and include, without limitation, anti-PD-L1 antibodies such as MEDI4736 (durvalumab; AstraZeneca; see WO 2011/066389), MSB-0010718C (see US 2014/0341917), YW243.55.S70 (see SEQ ID NO: 20 of WO 2010/077634 and U.S. Pat. No. 8,217,149), MIHI (Affymetrix eBioscience; cf. EP 3 230 319), MDX-1105 (Roche/Genentech; see WO2013019906 and U.S. Pat. No. 8,217,149) STI-1014 (Sorrento; see WO2013/181634), CK-301 (Checkpoint Therapeutics), KN035 (3D Med/Alphamab; see Zhang et al., 2017, Cell Discov. 3:17004), atezolizumab (TECENTRIQ; RG7446; MPDL3280A; R05541267; see U.S. Pat. No. 9,724,413), BMS-936559 (Bristol Myers Squibb; see U.S. Pat. No. 7,943,743, WO 2013/173223), avelumab (bavencio; cf. US 2014/0341917), LY3300054 (Eli Lilly Co.), CX-072 (Proclaim-CX-072; also called CytomX; see WO2016/149201), FAZ053, KN035 (see WO2017020801 and WO2017020802), MDX-1105 (see US 2015/0320859), anti-PD-L1 antibodies disclosed in U.S. Pat. No. 7,943,743, including 3G10, 12A4 (also referred to as BMS-936559), 10A5, 5F8, 10H10, 1B12, 7H1, 11E6, 12B7, and 13G4, anti-PD-L1 antibodies as described in WO 2010/077634, U.S. Pat. No. 8,217,149, WO 2010/036959, WO 2010/077634, WO 2011/066342, U.S. Pat. Nos. 8,217,149, 7,943,743, WO 2010/089411, U.S. Pat. Nos. 7,635,757, 8,217,149, US 2009/0317368, WO 2011/066389, WO2017/034916, WO2017/020291, WO2017/020858, WO2017/020801, WO2016/111645, WO2016/197367, WO2016/061142, WO2016/149201, WO2016/000619, WO2016/160792, WO2016/022630, WO2016/007235, WO2015/179654, WO2015/173267, WO2015/181342, WO2015/109124, WO 2018/222711, WO2015/112805, WO2015/061668, WO2014/159562, WO2014/165082, WO2014/100079.

Checkpoint inhibitors may be administered in any manner and by any route known in the art. The mode and route of administration will depend on the type of checkpoint inhibitor to be used.

Checkpoint inhibitors may be administered in the form of any suitable pharmaceutical composition as described herein.

Checkpoint inhibitors may be administered in the form of nucleic acid, such DNA or RNA molecules, encoding an immune checkpoint inhibitor, e.g., an inhibitory nucleic acid molecule or an antibody or fragment thereof. For example, antibodies can be delivered encoded in expression vectors, as described herein. Nucleic acid molecules can be delivered as such, e.g., in the form of a plasmid or mRNA molecule, or complexed with a delivery vehicle, e.g., a liposome, lipoplex or nucleic-acid lipid particles. Checkpoint inhibitors may also be administered via an oncolytic virus comprising an expression cassette encoding the checkpoint inhibitor. Checkpoint inhibitors may also be administered by administration of endogeneic or allogeneic cells able to express a checkpoint inhibitor, e.g., in the form of a cell based therapy.

The term “cell based therapy” refers to the transplantation of cells (e.g., T lymphocytes, dendritic cells, or stem cells) expressing an immune checkpoint inhibitor into a subject for the purpose of treating a disease or disorder (e.g., a cancer disease). In one embodiment, the cell based therapy comprises genetically engineered cells. In one embodiment, the genetically engineered cells express an immune checkpoint inhibitor, such as described herein. In one embodiment, the genetically engineered cells express an immune checkpoint inhibitor that is an inhibitory nucleic acid molecule, such as a siRNA, shRNA, an oligonucleotide, antisense DNA or RNA, an aptamer, an antibody or a fragment thereof or a soluble immune checkpoint protein or fusion. Genetically engineered cells may also express further agents that enhance T cell function. Such agents are known in the art. Cell based therapies for the use in inhibition of immune checkpoint signaling are disclosed, e.g., in WO 2018/222711, herein incorporated by reference in its entirety.

The term “oncolytic virus” as used herein, refers to a virus capable of selectively replicating in and slowing the growth or inducing the death of a cancerous or hyperproliferative cell, either in vitro or in vivo, while having no or minimal effect on normal cells. An oncolytic virus for the delivery of an immune checkpoint inhibitor comprises an expression cassette that may encode an immune checkpoint inhibitor that is an inhibitory nucleic acid molecule, such as a siRNA, shRNA, an oligonucleotide, antisense DNA or RNA, an aptamer, an antibody or a fragment thereof or a soluble immune checkpoint protein or fusion. The oncolytic virus preferably is replication competent and the expression cassette is under the control of a viral promoter, e.g., synthetic early/late poxvirus promoter. Exemplary oncolytic viruses include vesicular stomatitis virus (VSV), rhabdoviruses (e.g., picornaviruses such as Seneca Valley virus; SVV-001), coxsackievirus, parvovirus, Newcastle disease virus (NDV), herpes simplex virus (HSV; OncoVEX GMCSF), retroviruses (e.g., influenza viruses), measles virus, reovirus, Sinbis virus, vaccinia virus, as exemplarily described in WO 2017/209053 (including Copenhagen, Western Reserve, Wyeth strains), and adenovirus (e.g., Delta-24, Delta-24-RGD, ICOVIR-5, ICOVIR-7, Onyx-015, ColoAd1, H101, AD5/3-D24-GMCSF). Generation of recombinant oncolytic viruses comprising a soluble form of an immune checkpoint inhibitor and methods for their use are disclosed in WO 2018/022831, herein incorporated by reference in its entirety. Oncolytic viruses can be used as attenuated viruses.

As described herein, an anti-CLDN18.2 antibody is administered together, i.e., co-administered, with a checkpoint inhibitor to a subject, e.g., a patient. In certain embodiments, the checkpoint inhibitor and the anti-CLDN18.2 antibody are administered as a single composition to the subject. In certain embodiments, the checkpoint inhibitor and the anti-CLDN18.2 antibody are administered concurrently (as separate compositions at the same time) to the subject. In certain embodiments, the checkpoint inhibitor and the anti-CLDN18.2 antibody are administered separately to the subject. In certain embodiments, the checkpoint inhibitor is administered before the anti-CLDN18.2 antibody to the subject. In certain embodiments, the checkpoint inhibitor is administered after the anti-CLDN18.2 antibody to the subject. In certain embodiments, the checkpoint inhibitor and the anti-CLDN18.2 antibody are administered to the subject on the same day. In certain embodiments, the checkpoint inhibitor and the anti-CLDN18.2 antibody are administered to the subject on different days.

According to the invention, the term “chemotherapeutic agent” includes cytotoxic agents, cytostatic agents or combinations thereof. Chemotherapeutic agents may affect cells in one of the following ways: (1) damage the DNA of the cells so they can no longer reproduce, (2) inhibit the synthesis of new DNA strands so that no cell replication is possible, (3) stop the mitotic processes of the cells so that the cells cannot divide into two cells. A chemotherapeutic agent may be an agent stabilizing or increasing expression of CLDN18.2.

The term “agent stabilizing or increasing expression of CLDN18.2” refers to an agent or a combination of agents the provision of which to cells results in increased RNA and/or protein levels of CLDN18.2, preferably in increased levels of CLDN18.2 protein on the cell surface, compared to the situation where the cells are not provided with the agent or the combination of agents. Preferably, the cell is a cancer cell, in particular a cancer cell expressing CLDN18.2, such as a cell of the cancer types described herein. The term “agent stabilizing or increasing expression of CLDN18.2” refers, in particular, to an agent or a combination of agents the provision of which to cells results in a higher density of CLDN18.2 on the surface of said cells compared to the situation where the cells are not provided with the agent or the combination of agents. “Stabilizing expression of CLDN18.2” includes, in particular, the situation where the agent or the combination of agents prevents a decrease or reduces a decrease in expression of CLDN18.2, e.g., expression of CLDN18.2 would decrease without provision of the agent or the combination of agents and provision of the agent or the combination of agents prevents said decrease or reduces said decrease of CLDN18.2 expression. “Increasing expression of CLDN18.2” includes, in particular, the situation where the agent or the combination of agents increases expression of CLDN18.2, e.g., expression of CLDN18.2 would decrease, remain essentially constant or increase without provision of the agent or the combination of agents and provision of the agent or the combination of agents increases CLDN18.2 expression compared to the situation without provision of the agent or the combination of agents so that the resulting expression is higher compared to the situation where expression of CLDN18.2 would decrease, remain essentially constant or increase without provision of the agent or the combination of agents.

According to the invention, the term “agent stabilizing or increasing expression of CLDN18.2” preferably relates to an agent or a combination of agents such a cytostatic compound or a combination of cytostatic compounds the provision of which to cells, in particular cancer cells, results in the cells being arrested in or accumulating in one or more phases of the cell cycle, preferably in one or more phases of the cell cycle other than the G1- and G0-phases, preferably other than the G1-phase, preferably in one or more of the G2- or S-phase of the cell cycle such as the G1/G2-, S/G2-, G2- or S-phase of the cell cycle. The term “cells being arrested in or accumulating in one or more phases of the cell cycle” means that the percentage of cells which are in said one or more phases of the cell cycle increases. Each cell goes through a cycle comprising four phases in order to replicate itself. The first phase called G1 is when the cell prepares to replicate its chromosomes. The second stage is called S, and in this phase DNA synthesis occurs and the DNA is duplicated. The next phase is the G2 phase, when the RNA and protein duplicate. The final stage is the M stage, which is the stage of actual cell division. In this final stage, the duplicated DNA and RNA split and move to separate ends of the cell, and the cell actually divides into two identical, functional cells. Chemotherapeutic agents which are DNA damaging agents usually result in an accumulation of cells in the G1 and/or G2 phase. Chemotherapeutic agents which block cell growth by interfering with DNA synthesis such as antimetabolites usually result in an accumulation of cells in the S-phase. Examples of these drugs are 6-mercaptopurine and 5-fluorouracil.

According to the invention, the term “agent stabilizing or increasing expression of CLDN18.2” includes platinum compounds such as oxaliplatin and cisplatin, and nucleoside analogs such as 5-fluorouracil or prodrugs thereof, and combinations of drugs such as combinations of drugs comprising oxaliplatin and 5-fluorouracil.

In one preferred embodiment, a “chemotherapeutic agent” is an “agent inducing immunogenic cell death”.

In specific circumstances, cancer cells can enter a lethal stress pathway linked to the emission of a spatiotemporally defined combination of signals that is decoded by the immune system to activate tumor-specific immune responses (Zitvogel L. et al. (2010) Cell 140: 798-804). In such scenario cancer cells are triggered to emit signals that are sensed by innate immune effectors such as dendritic cells to trigger a cognate immune response that involves CD8+ T cells and IFN-γ signalling so that tumor cell death may elicit a productive anticancer immune response. These signals include the pre-apoptotic exposure of the endoplasmic reticulum (ER) chaperon calreticulin (CRT) at the cell surface, the pre-apoptotic secretion of ATP, and the post-apoptotic release of the nuclear protein HMGB1. Together, these processes constitute the molecular determinants of immunogenic cell death (ICD). Anthracyclines, oxaliplatin, and γ irradiation are able to induce all signals that define ICD, while cisplatin, for example, which is deficient in inducing CRT translocation from the ER to the surface of dying cells—a process requiring ER stress—requires complementation by thapsigargin, an ER stress inducer.

According to the invention, the term “agent inducing immunogenic cell death” refers to an agent or a combination of agents which when provided to cells, in particular cancer cells, is capable of inducing the cells to enter a lethal stress pathway which finally results in tumor-specific immune responses. In particular, an agent inducing immunogenic cell death when provided to cells induces the cells to emit a spatiotemporally defined combination of signals, including, in particular, the pre-apoptotic exposure of the endoplasmic reticulum (ER) chaperon calreticulin (CRT) at the cell surface, the pre-apoptotic secretion of ATP, and the post-apoptotic release of the nuclear protein HMGB1.

According to the invention, the term “agent inducing immunogenic cell death” includes oxaliplatin.

According to the invention, the term “platinum compound” refers to compounds containing platinum in their structure such as platinum complexes. In particular, this term refers to such compounds as used in platinum based chemotherapy and includes compounds such as cisplatin, carboplatin and oxaliplatin.

The term “cisplatin” or “cisplatinum” refers to the compound cis-diamminedichloroplatinum(II) (CDDP) of the following formula:

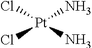

The term “carboplatin” refers to the compound cis-diammine(1,1-cyclobutanedicarboxylato)platinum(II) of the following formula:

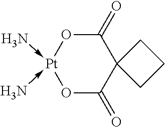

The term “oxaliplatin” refers to a compound which is a platinum compound that is complexed to a diaminocyclohexane carrier ligand of the following formula:

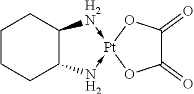

In particular, the term “oxaliplatin” refers to the compound [(1R,2R)-cyclohexane-1,2-diamine](ethanedioato-O,O′)platinum(II). Oxaliplatin for injection is also marketed under the trade name Eloxatine.

The term “nucleoside analog” refers to a structural analog of a nucleoside, a category that includes both purine analogs and pyrimidine analogs. In particular, the term “nucleoside analog” refers to such compounds as used in antimetabolite chemotherapy and includes fluoropyrimidine derivatives and precursors thereof which includes fluorouracil and prodrugs thereof including, without limitation, fluorouracil (5-FU), capecitabine, floxuridine, and tegafur. The term “antimetabolite chemotherapy” refers to the use of an agent which is structurally similar to a metabolite, but cannot be used by the body in a productive manner. In certain embodiments, antimetabolite chemotherapy interferes with the production of nucleic acids, RNA and DNA.

The term “fluorouracil” or “5-fluorouracil” (5-FU or f5U) (sold under the brand names Adrucil, Carac, Efudix, Efudex and Fluoroplex) is a compound which is a pyrimidine analog of the following formula:

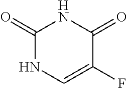

In particular, the term refers to the compound 5-fluoro-1H-pyrimidine-2,4-dione.

The term “capecitabine” (Xeloda, Roche) refers to a chemotherapeutic agent that is a prodrug that is converted into 5-FU in the tissues. Capecitabine which may be orally administered has the following formula:

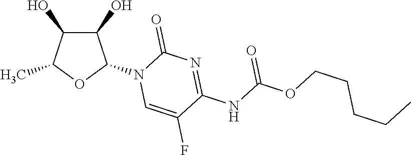 [SMILES: CCCCCOC(=O)Nc1nc(=O)n([C@@H]2O[C@H](C)[C@@H](O)[C@H]2O)cc1F]

In particular, the term refers to the compound pentyl [1-(3,4-dihydroxy-5-methyltetrahydrofuran-2-yl)-5-fluoro-2-oxo-1H-pyrimidin-4-yl]carbamate.

“Floxuridine” (5-fluorodeoxyuridine) is an oncology drug that is rapidly catabolized to 5-fluorouracil, which is the active form of the drug. Floxuridine has the following formula:

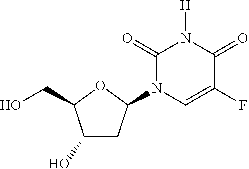 [SMILES: O=c1[nH]c(=O)n([C@H]2C[C@H](O)[C@@H](CO)O2)cc1F]

“Tegafur” (5-fluoro-1-(oxolan-2-yl)pyrimidine-2,4-dione) is a chemotherapeutic prodrug of 5-fluorouracil. When metabolised, it becomes 5-fluorouracil. Tegafur has the following formula:

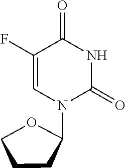

The term “doxifluridine” (5′-deoxy-5-fluorouridine) is a fluoropyrimidine derivative of 5-fluorouracil. This second generation nucleoside analog prodrug is used as a cytostatic agent in chemotherapy in several Asian countries including China and South Korea. Within a cell, pyrimidine nucleoside phosphorylase or thymidine phosphorylase can metabolize doxifluridine into 5-fluorouracil. It is also a metabolite of capecitabine. Doxifluridine which may be orally administered has the following formula:

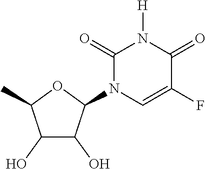 [SMILES: C[C@H]1O[C@@H](n2cc(F)c(=O)[nH]c2=O)C(O)C1O]

The term “Carmofur” (INN) or “HCFU” refers to 1-hexylcarbamoyl-5-fluorouracil:

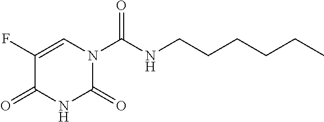 [SMILES: CCCCCCNC(=O)n1cc(F)c(=O)[nH]c1=O]

This compound is a pyrimidine analogue used as an antineoplastic agent. It is a derivative of fluorouracil, being a lypophilic-masked analog of 5-fluorouracil. Once inside a cell, carmofur prodrug is converted into 5-fluorouracil.

The present invention may comprise the administration of a platinum compound and a fluoropyrimidine compound or precursor thereof as part of a chemotherapeutic regimen established in cancer treatment. Such chemotherapeutic regimen may be selected from the group consisting of EOX chemotherapy, ECF chemotherapy, ECX chemotherapy, EOF chemotherapy, FLO chemotherapy, CAPOX chemotherapy, FOLFOX chemotherapy, DCF chemotherapy, SOX chemotherapy and FLOT chemotherapy.

The drug combination used in EOX chemotherapy comprises epirubicin, oxaliplatin and capecitabine. The drug combination used in ECF chemotherapy comprises epirubicin, cisplatin and 5-fluorouracil. The drug combination used in ECX chemotherapy comprises epirubicin, cisplatin and capecitabine. The drug combination used in EOF chemotherapy comprises epirubicin, oxaliplatin and 5-fluorouracil. The drug combination used in FLO chemotherapy comprises of 5-fluorouracil, folinic acid and oxaliplatin. The drug combination used in SOX chemotherapy comprises tegafur, gimeracil, oteracil and oxaliplatin.

FOLFOX is a chemotherapy regimen made up of folinic acid (leucovorin), 5-fluorouracil and oxaliplatin. The recommended dose schedule given every two weeks is as follows: Day 1: Oxaliplatin 85 mg/m2 IV infusion and leucovorin 200 mg/m2 IV infusion, followed by 5-FU 400 mg/m2 IV bolus, followed by 5-FU 600 mg/m2 IV infusion as a 22-hour continuous infusion; Day 2: Leucovorin 200 mg/m2 IV infusion over 120 minutes, followed by 5-FU 400 mg/m2 IV bolus given over 2-4 minutes, followed by 5-FU 600 mg/m2 IV infusion as a 22-hour continuous infusion.

There are several different FOLFOX regimens that differ in the doses and ways in which the three drugs are given.

In one embodiment, the chemotherapy regimen is a modified FOLFOX-6 regimen (mFOLFOX6). In one embodiment, the mFOLFOX6 regimen comprises 85 mg/m2 oxaliplatin, 400 mg/m2 bolus of 5-FU, and 400 mg/m2 leucovorin, followed by 2,400 mg/m2 of 5-FU as a continuous infusion.

In one embodiment, doses and modes of administration of mFOLFOX6 treatment are as follows: Oxaliplatin 85 mg/m2 IV infusion, e.g., a 2-hour IV infusion in e.g., 500 mL, concurrent with leucovorin 400 mg/m2 (or levo-leucovorin [levofolinate or levo-folinic acid] 200 mg/m2) IV infusion. This is followed by 5-FU 400 mg/m2 IV bolus (e.g., given in 5 to 15 minutes), followed by a continuous 5-FU infusion 2400 mg/m2, e.g., over 46 to 48-hours.

mFOLFOX6 may be repeated every 2 weeks [days 15 and 29]. A cycle may comprise 3 treatments and may last 6 weeks. In one embodiment, subjects receive up to 12 mFOLFOX6 treatments (4 cycles). According to the present invention, mFOLFOX6 treatment may follow administration of the anti-CLDN18.2 antibody and administration of an anti-PD-1 antibody.

In one embodiment, a treatment described herein may comprise the following:

Anti-CLDN18.2 antibody loading dose of 800 mg/m2 (or 600 mg/m2) in combination with nivolumab 240 mg and mFOLFOX6 on cycle 1 day 1, followed by anti-CLDN18.2 antibody 400 mg/m2 in combination with nivolumab 240 mg and mFOLFOX6 every 2 weeks [days 15 and 29] (1 cycle=6 weeks). Anti-CLDN18.2 antibody may be administered first, followed by nivolumab and then mFOLFOX6. In one embodiment, subjects receive up to 12 mFOLFOX6 treatments (4 cycles). Beginning at cycle 5, subjects may continue on 5-FU and leucovorin or folinic acid along with anti-CLDN18.2 antibody and nivolumab. In a certain embodiment, nivolumab is administered intravenously, e.g, over 30 minutes, on day 1 of every 2 week cycle and will be infused after the infusion of the anti-CLDN18.2 antibody is completed, e.g., 1 hour after the infusion of the anti-CLDN18.2 antibody is completed.

The drug combination used in CAPOX chemotherapy comprises of capecitabine and oxaliplatin. CAPOX regime operates in 3-week cycles, usually with 8 cycles in total; capecitabine is orally taken for twice daily for two weeks, while oxaliplatin is administrated by IV on the first day of the cycle; there is a one-week rest period before the next cycle.

The drug combination used in DCF chemotherapy comprises of docetaxel, cisplatin and 5-fluorouracil.

The drug combination used in FLOT chemotherapy comprises of docetaxel, oxaliplatin, 5-fluorouracil and folinic acid.

The term “folinic acid” or “leucovorin” refers to a compound useful in synergistic combination with the chemotherapy agent 5-fluorouracil. Folinic acid has the following formula:

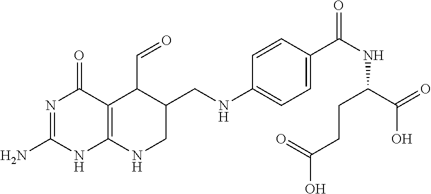 [SMILES: Nc1nc(=O)c2c([nH]1)NCC(CNc1ccc(C(=O)N[C@@H](CCC(=O)O)C(=O)O)cc1)C2C=O]

In particular, the term refers to the compound (2S)-2-{[4-[(2-amino-5-formyl-4-oxo-5,6,7,8-tetrahydro-1H-pteridin-6-yl)methylamino]benzoyl]amino}pentanedioic acid.

The term “antigen” relates to an agent such as a protein or peptide comprising an epitope against which an immune response is directed and/or is to be directed. In a preferred embodiment, an antigen is a tumor-associated antigen, such as CLDN18.2, i.e., a constituent of cancer cells which may be derived from the cytoplasm, the cell surface and the cell nucleus, in particular those antigens which are produced, preferably in large quantity, intracellularly or as surface antigens on cancer cells.

In the context of the present invention, the term “tumor-associated antigen” preferably relates to proteins that are under normal conditions specifically expressed in a limited number of tissues and/or organs or in specific developmental stages and are expressed or aberrantly expressed in one or more tumor or cancer tissues. In the context of the present invention, the tumor-associated antigen is preferably associated with the cell surface of a cancer cell and is preferably not or only rarely expressed in normal tissues.

The term “epitope” refers to an antigenic determinant in a molecule, i.e., to the part in a molecule that is recognized by the immune system, for example, that is recognized by an antibody. For example, epitopes are the discrete, three-dimensional sites on an antigen, which are recognized by the immune system. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. An epitope of a protein such as CLDN18.2 preferably comprises a continuous or discontinuous portion of said protein and is preferably between 5 and 100, preferably between 5 and 50, more preferably between 8 and 30, most preferably between 10 and 25 amino acids in length, for example, the epitope may be preferably 8, 9, 10, 11, 12, 13, 14, 15, 16, 17, 18, 19, 20, 21, 22, 23, 24, or 25 amino acids in length.

The term “antibody” refers to a glycoprotein comprising at least two heavy (H) chains and two light (L) chains inter-connected by disulfide bonds, and includes any molecule comprising an antigen binding portion thereof. The term “antibody” includes monoclonal antibodies and fragments or derivatives of antibodies, including, without limitation, human antibodies, humanized antibodies, chimeric antibodies, single chain antibodies, e.g., scFv's and antigen-binding antibody fragments such as Fab and Fab′ fragments and also includes all recombinant forms of antibodies, e.g., antibodies expressed in prokaryotes, unglycosylated antibodies, and any antigen-binding antibody fragments and derivatives as described herein. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The antibodies described herein may be human antibodies. The term “human antibody”, as used herein, is intended to include antibodies having variable and constant regions derived from human germline immunoglobulin sequences. The human antibodies described herein may include amino acid residues not encoded by human germline immunoglobulin sequences (e.g., mutations introduced by random or site-specific mutagenesis in vitro or by somatic mutation in vivo).

The term “humanized antibody” refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen binding sites may be wild-type or modified by one or more amino acid substitutions, e.g., modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

The term “chimeric antibody” refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another. Typically the variable region of both light and heavy chains mimics the variable regions of antibodies derived from one species of mammals, while the constant portions are homologous to sequences of antibodies derived from another. One clear advantage to such chimeric forms is that the variable region can conveniently be derived from presently known sources using readily available B-cells or hybridomas from non-human host organisms in combination with constant regions derived from, for example, human cell preparations. While the variable region has the advantage of ease of preparation and the specificity is not affected by the source, the constant region being human, is less likely to elicit an immune response from a human subject when the antibodies are injected than would the constant region from a non human source. However the definition is not limited to this particular example.

The terms “antigen-binding portion” of an antibody (or simply “binding portion”) or “antigen-binding fragment” of an antibody (or simply “binding fragment”) or similar terms refer to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) Fab fragments, monovalent fragments consisting of the VL, VH, CL and CH domains; (ii) F(ab′)2 fragments, bivalent fragments comprising two Fab fragments linked by a disulfide bridge at the hinge region; (iii) Fd fragments consisting of the VH and CH domains; (iv) Fv fragments consisting of the VL and VH domains of a single arm of an antibody, (v) dAb fragments (Ward et al., (1989) Nature 341: 544-546), which consist of a VH domain; (vi) isolated complementarity determining regions (CDR), and (vii) combinations of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding fragment” of an antibody. A further example is binding-domain immunoglobulin fusion proteins comprising (i) a binding domain polypeptide that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region. The binding domain polypeptide can be a heavy chain variable region or a light chain variable region. The binding-domain immunoglobulin fusion proteins are further disclosed in US 2003/0118592 and US 2003/0133939. These antibody fragments are obtained using conventional techniques known to those with skill in the art, and the fragments are screened for utility in the same manner as are intact antibodies.

The term “bispecific molecule” is intended to include any agent, e.g., a protein, peptide, or protein or peptide complex, which has two different binding specificities. For example, the molecule may bind to, or interact with (a) a cell surface antigen, and (b) an Fc receptor on the surface of an effector cell. The term “multispecific molecule” or “heterospecific molecule” is intended to include any agent, e.g., a protein, peptide, or protein or peptide complex, which has more than two different binding specificities. For example, the molecule may bind to, or interact with (a) a cell surface antigen, (b) an Fc receptor on the surface of an effector cell, and (c) at least one other component. Accordingly, the invention includes, but is not limited to, bispecific, trispecific, tetraspecific, and other multispecific molecules which are directed to CLDN18.2, and to other targets, such as Fc receptors on effector cells. The term “bispecific antibodies” also includes multivalent antibodies, such as trivalent antibodies with two different binding specificities, tetravalent antibodies with two or three different binding specificities, and so on. The term “bispecific antibodies” also includes diabodies. Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90: 6444-6448; Poljak, R. J., et al. (1994) Structure 2: 1121-1123).

An antibody may be conjugated to a therapeutic moiety or agent, such as a cytotoxin, a drug (e.g., an immunosuppressant) or a radioisotope. A cytotoxin or cytotoxic agent includes any agent that is detrimental to and, in particular, kills cells. Examples include maytansins (e.g., mertansine, ravtansine or emtanside), auristatins (Monomethyl auristatin F (MMAF), Monomethyl auristatin E (MMAE)), maytansinoid (DM1 or DM4), dolastatins, calicheamicins (e.g., ozogamicin), pyrrolobenzidiazepine dimers (e.g., tesirine, tairine), duocarmycins (e.g., Duocarmycin SA, CC-1065, duocarmazine) and α-amanitin, irinotecan or its derivative SN-38, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof, antimetabolites (e.g., methotrexate, 6-mercaptopurine, 6-thioguanine, cytarabine, fludarabin, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC), and anti-mitotic agents (e.g., vincristine and vinblastine). In a preferred embodiment, the therapeutic agent is a cytotoxic agent or a radiotoxic agent. In another embodiment, the therapeutic agent is an immunosuppressant. In yet another embodiment, the therapeutic agent is GM-CSF. In a preferred embodiment, the therapeutic agent is doxorubicin, cisplatin, bleomycin, sulfate, carmustine, chlorambucil, cyclophosphamide or ricin A.

Antibodies also can be conjugated to a radioisotope, e.g., iodine-131, yttrium-90 or indium-111, to generate cytotoxic radiopharmaceuticals.

The antibody conjugates of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62: 119-58 (1982).

As used herein, an antibody is “derived from” a particular germline sequence if the antibody is obtained from a system by immunizing an animal or by screening an immunoglobulin gene library, and wherein the selected antibody is at least 90%, more preferably at least 95%, even more preferably at least 96%, 97%, 98%, or 99% identical in amino acid sequence to the amino acid sequence encoded by the germline immunoglobulin gene. Typically, an antibody derived from a particular germline sequence will display no more than 10 amino acid differences, more preferably, no more than 5, or even more preferably, no more than 4, 3, 2, or 1 amino acid difference from the amino acid sequence encoded by the germline immunoglobulin gene.

As used herein, the term “heteroantibodies” refers to two or more antibodies, derivatives thereof, or antigen binding regions linked together, at least two of which have different specificities. These different specificities include a binding specificity for an Fc receptor on an effector cell, and a binding specificity for an antigen or epitope on a target cell, e.g., a tumor cell.

The antibodies described herein may be monoclonal antibodies. The term “monoclonal antibody” as used herein refers to a preparation of antibody molecules of single molecular composition. A monoclonal antibody displays a single binding specificity and affinity. In one embodiment, the monoclonal antibodies are produced by a hybridoma which includes a B cell obtained from a non-human animal, e.g., mouse, fused to an immortalized cell.

The antibodies described herein may be recombinant antibodies. The term “recombinant antibody”, as used herein, includes all antibodies that are prepared, expressed, created or isolated by recombinant means, such as (a) antibodies isolated from an animal (e.g., a mouse) that is transgenic or transchromosomal with respect to the immunoglobulin genes or a hybridoma prepared therefrom, (b) antibodies isolated from a host cell transformed to express the antibody, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

Antibodies described herein may be derived from different species, including but not limited to mouse, rat, rabbit, guinea pig and human.

Antibodies described herein include polyclonal and monoclonal antibodies and include IgA such as IgA1 or IgA2, IgG1, IgG2, IgG3, IgG4, IgE, IgM, and IgD antibodies. In various embodiments, the antibody is an IgG1 antibody, more particularly an IgG1, kappa or IgG1, lambda isotype (e.g. IgG1, κ, λ), an IgG2a antibody (e.g., IgG2a, κ, λ), an IgG2b antibody (e.g., IgG2b, κ, λ), an IgG3 antibody (e.g., IgG3, κ, λ) or an IgG4 antibody (e.g., IgG4, κ. λ).

The term “transfectoma”, as used herein, includes recombinant eukaryotic host cells expressing an antibody, such as CHO cells, NS/0 cells, HEK293 cells, HEK293T cells, plant cells, or fungi, including yeast cells.

As used herein, a “heterologous antibody” is defined in relation to a transgenic organism producing such an antibody. This term refers to an antibody having an amino acid sequence or an encoding nucleic acid sequence corresponding to that found in an organism not consisting of the transgenic organism and being generally derived from a species other than the transgenic organism.

As used herein, a “heterohybrid antibody” refers to an antibody having light and heavy chains of different organismal origins. For example, an antibody having a human heavy chain associated with a murine light chain is a heterohybrid antibody.

The invention includes all antibodies and derivatives of antibodies as described herein which for the purposes of the invention are encompassed by the term “antibody”. The term “antibody derivatives” refers to any modified form of an antibody, e.g., a conjugate of the antibody and another agent or antibody, or an antibody fragment.

The antibodies described herein are preferably isolated. “Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell. An “isolated antibody” as used herein, is intended to include an antibody which is substantially free of other antibodies having different antigenic specificities (e.g., an isolated antibody that specifically binds to CLDN18.2 is substantially free of antibodies that specifically bind antigens other than CLDN18.2). An isolated antibody that specifically binds to an epitope, isoform or variant of human CLDN18.2 may, however, have cross-reactivity to other related antigens, e.g., from other species (e.g., CLDN18.2 species homologs). Moreover, an isolated antibody may be substantially free of other cellular material and/or chemicals. In one embodiment of the invention, a combination of “isolated” monoclonal antibodies relates to antibodies having different specificities and being combined in a well defined composition or mixture.

The term “binding” according to the invention preferably relates to a specific binding.

According to the present invention, an antibody is capable of binding to a predetermined target if it has a significant affinity for said predetermined target and binds to said predetermined target in standard assays. “Affinity” or “binding affinity” is often measured by equilibrium dissociation constant (K_{D}). Preferably, the term “significant affinity” refers to the binding to a predetermined target with a dissociation constant (K_{D}) of 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, or 10⁻¹² M or lower.

An antibody is not (substantially) capable of binding to a target if it has no significant affinity for said target and does not bind significantly, in particular does not bind detectably, to said target in standard assays. Preferably, the antibody does not detectably bind to said target if present in a concentration of up to 2, preferably 10, more preferably 20, in particular 50 or 100 μg/ml or higher. Preferably, an antibody has no significant affinity for a target if it binds to said target with a K_{D} that is at least 10-fold, 100-fold, 10³-fold, 10⁴-fold, 10⁵-fold, or 10⁶-fold higher than the K_{D} for binding to the predetermined target to which the antibody is capable of binding. For example, if the K_{D} for binding of an antibody to the target to which the antibody is capable of binding is 10⁻⁷ M, the K_{D} for binding to a target for which the antibody has no significant affinity would be is at least 10⁻⁶ M, 10⁻⁵ M, 10⁻⁴M, 10⁻³ M, 10⁻² M, or 10⁻¹ M.

An antibody is specific for a predetermined target if it is capable of binding to said predetermined target while it is not capable of binding to other targets, i.e., has no significant affinity for other targets and does not significantly bind to other targets in standard assays. According to the invention, an antibody is specific for CLDN18.2 if it is capable of binding to CLDN18.2 but is not (substantially) capable of binding to other targets. Preferably, an antibody is specific for CLDN18.2 if the affinity for and the binding to such other targets does not significantly exceed the affinity for or binding to CLDN18.2-unrelated proteins such as bovine serum albumin (BSA), casein, human serum albumin (HSA) or non-claudin transmembrane proteins such as MHC molecules or transferrin receptor or any other specified polypeptide. Preferably, an antibody is specific for a predetermined target if it binds to said target with a K_{D} that is at least 10-fold, 100-fold, 10³-fold, 10⁴-fold, 10⁵-fold, or 10⁶-fold lower than the K_{D} for binding to a target for which it is not specific. For example, if the K_{D} for binding of an antibody to the target for which it is specific is 10⁻⁷ M, the K_{D} for binding to a target for which it is not specific would be at least 10⁻⁶ M, 10⁻⁵ M, 10⁻⁴M, 10⁻³ M, 10⁻² M, or 10⁻¹ M.

Binding of an antibody to a target can be determined experimentally using any suitable method; see, for example, Berzofsky et al., “Antibody-Antigen Interactions” In Fundamental Immunology, Paul, W. E., Ed., Raven Press New York, N Y (1984), Kuby, Janis Immunology, W. H. Freeman and Company New York, N Y (1992), and methods described herein. Affinities may be readily determined using conventional techniques, such as by equilibrium dialysis; by using the BIAcore 2000 instrument, using general procedures outlined by the manufacturer; by radioimmunoassay using radiolabeled target antigen; or by another method known to the skilled artisan. The affinity data may be analyzed, for example, by the method of Scatchard et al., Ann N.Y. Acad. ScL, 51:660 (1949). The measured affinity of a particular antibody-antigen interaction can vary if measured under different conditions, e.g., salt concentration, pH. Thus, measurements of affinity and other antigen-binding parameters, e.g., K_{D}, IC₅₀, are preferably made with standardized solutions of antibody and antigen, and a standardized buffer.

As used herein, “isotype” refers to the antibody class (e.g., IgM or IgG1) that is encoded by heavy chain constant region genes.

As used herein, “isotype switching” refers to the phenomenon by which the class, or isotype, of an antibody changes from one Ig class to one of the other Ig classes.

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally occurring.

The term “rearranged” as used herein refers to a configuration of a heavy chain or light chain immunoglobulin locus wherein a V segment is positioned immediately adjacent to a D-J or J segment in a conformation encoding essentially a complete VH or VL domain, respectively. A rearranged immunoglobulin (antibody) gene locus can be identified by comparison to germline DNA; a rearranged locus will have at least one recombined heptamer/nonamer homology element.

The term “unrearranged” or “germline configuration” as used herein in reference to a V segment refers to the configuration wherein the V segment is not recombined so as to be immediately adjacent to a D or J segment.

According to the invention an anti-CLDN18.2 antibody is an antibody capable of binding to an epitope present in CLDN18.2, preferably an epitope located within the extracellular domains of CLDN18.2, in particular the first extracellular domain, preferably amino acid positions 29 to 78 of CLDN18.2. In particular embodiments, an anti-CLDN18.2 antibody is an antibody capable of binding to (i) an epitope on CLDN18.2 which is not present on CLDN18.1, preferably SEQ ID NO: 3, 4, and 5, (ii) an epitope localized on the CLDN18.2-loop1, preferably SEQ ID NO: 8, (iii) an epitope localized on the CLDN18.2-loop2, preferably SEQ ID NO: 10, (iv) an epitope localized on the CLDN18.2-loopD3, preferably SEQ ID NO: 11, (v) an epitope, which encompass CLDN18.2-loop1 and CLDN18.2-loopD3, or (vi) a non-glycosylated epitope localized on the CLDN18.2-loopD3, preferably SEQ ID NO: 9.

According to the invention an anti-CLDN18.2 antibody preferably is an antibody binding to CLDN18.2 but not to CLDN18.1. Preferably, an anti-CLDN18.2 antibody is specific for CLDN18.2. Preferably, an anti-CLDN18.2 antibody is an antibody binding to CLDN18.2 expressed on the cell surface. In particular preferred embodiments, an anti-CLDN18.2 antibody binds to native epitopes of CLDN18.2 present on the surface of living cells. Preferably, an anti-CLDN18.2 antibody binds to one or more peptides selected from the group consisting of SEQ ID NOs: 1, 3-11, 44, 46, and 48-50. Preferably, an anti-CLDN18.2 antibody is specific for the afore mentioned proteins, peptides or immunogenic fragments or derivatives thereof. An anti-CLDN18.2 antibody may be obtained by a method comprising the step of immunizing an animal with a protein or peptide comprising an amino acid sequence selected from the group consisting of SEQ ID NOs: 1, 3-11, 44, 46, and 48-50, or a nucleic acid or host cell expressing said protein or peptide. Preferably, the antibody binds to cancer cells, in particular cells of the cancer types mentioned above and, preferably, does not bind substantially to non-cancerous cells.

Preferably, binding of an anti-CLDN18.2 antibody to cells expressing CLDN18.2 induces or mediates killing of cells expressing CLDN18.2. The cells expressing CLDN18.2 are preferably cancer cells and are, in particular, selected from the group consisting of tumorigenic gastric, esophageal, pancreatic, lung, ovarian, colon, hepatic, head-neck, and gallbladder cancer cells. Preferably, the antibody induces or mediates killing of cells by inducing one or more of complement dependent cytotoxicity (CDC) mediated lysis, antibody dependent cellular cytotoxicity (ADCC) mediated lysis, apoptosis, and inhibition of proliferation of cells expressing CLDN18.2. Preferably, ADCC mediated lysis of cells takes place in the presence of effector cells, which in particular embodiments are selected from the group consisting of monocytes, mononuclear cells, NK cells and PMNs. Inhibiting proliferation of cells can be measured in vitro by determining proliferation of cells in an assay using bromodeoxyuridine (5-bromo-2-deoxyuridine, BrdU). BrdU is a synthetic nucleoside which is an analogue of thymidine and can be incorporated into the newly synthesized DNA of replicating cells (during the S phase of the cell cycle), substituting for thymidine during DNA replication. Detecting the incorporated chemical using, for example, antibodies specific for BrdU indicates cells that were actively replicating their DNA.

In preferred embodiments, antibodies described herein can be characterized by one or more of the following properties:
a) specificity for CLDN18.2;
b) a binding affinity to CLDN18.2 of about 100 nM or less, preferably, about 5-10 nM or less and, more preferably, about 1-3 nM or less,
c) the ability to induce or mediate CDC on CLDN18.2 positive cells;
d) the ability to induce or mediate ADCC on CLDN18.2 positive cells;
e) the ability to inhibit the growth of CLDN18.2 positive cells;
f) the ability to induce apoptosis of CLDN18.2 positive cells.

In a particularly preferred embodiment, an anti-CLDN18.2 antibody is produced by a hybridoma deposited at the DSMZ (Mascheroder Weg 1b, 31824 Braunschweig, Germany; new address: Inhoffenstr. 7B, 31824 Braunschweig, Germany) and having the following designation and accession number:
a. 182-D1106-055, accession no. DSM ACC2737, deposited on Oct. 19, 2005
b. 182-D1106-056, accession no. DSM ACC2738, deposited on Oct. 19, 2005
c. 182-D1106-057, accession no. DSM ACC2739, deposited on Oct. 19, 2005
d. 182-D1106-058, accession no. DSM ACC2740, deposited on Oct. 19, 2005
e. 182-D1106-059, accession no. DSM ACC2741, deposited on Oct. 19, 2005
f. 182-D1106-062, accession no. DSM ACC2742, deposited on Oct. 19, 2005,
g. 182-D1106-067, accession no. DSM ACC2743, deposited on Oct. 19, 2005
h. 182-D758-035, accession no. DSM ACC2745, deposited on Nov. 17, 2005
i. 182-D758-036, accession no. DSM ACC2746, deposited on Nov. 17, 2005
j. 182-D758-040, accession no. DSM ACC2747, deposited on Nov. 17, 2005
k. 182-D1106-061, accession no. DSM ACC2748, deposited on Nov. 17, 2005
l. 182-D1106-279, accession no. DSM ACC2808, deposited on Oct. 26, 2006
m. 182-D1106-294, accession no. DSM ACC2809, deposited on Oct. 26, 2006,
n. 182-D1106-362, accession no. DSM ACC2810, deposited on Oct. 26, 2006.

Preferred antibodies according to the invention are those produced by and obtainable from the above-described hybridomas; i.e., 37G11 in the case of 182-D1106-055, 37H8 in the case of 182-D1106-056, 38G5 in the case of 182-D1106-057, 38H3 in the case of 182-D1106-058, 39F11 in the case of 182-D1106-059, 43A11 in the case of 182-D1106-062, 61C2 in the case of 182-D1106-067, 26B5 in the case of 182-D758-035, 26D12 in the case of 182-D758-036, 28D10 in the case of 182-D758-040, 42E12 in the case of 182-D1106-061, 125E1 in the case of 182-D1106-279, 163E12 in the case of 182-D1106-294, and 175D10 in the case of 182-D1106-362; and the chimerized and humanized forms thereof.

Preferred chimerized antibodies and their sequences are shown in the following table.

| | | | | | chimerized |
|---|---|---|---|---|---|
| | clone | mAb | Isotype | variable region | antibody |
| heavy | 43A11 | 182-D1106-062 | IgG2a | SEQ ID NO: 29 | SEQ ID NO: 14 |
| chain | 163E12 | 182-D1106-294 | IgG3 | SEQ ID NO: 30 | SEQ ID NO: 15 |
| | 125E1 | 182-D1106-279 | IgG2a | SEQ ID NO: 31 | SEQ ID NO: 16 |
| | 166E2 | 182-D1106-308 | IgG3 | SEQ ID NO: 33 | SEQ ID NO: 18 |
| | 175D10 | 182-D1106-362 | IgG1 | SEQ ID NO: 32 | SEQ ID NO: 17 |
| | 45C1 | 182-D758-187 | IgG2a | SEQ ID NO: 34 | SEQ ID NO: 19 |
| light | 43A11 | 182-D1106-062 | IgK | SEQ ID NO: 36 | SEQ ID NO: 21 |
| chain | 163E12 | 182-D1106-294 | IgK | SEQ ID NO: 35 | SEQ ID NO: 20 |
| | 125E1 | 182-D1106-279 | IgK | SEQ ID NO: 37 | SEQ ID NO: 22 |
| | 166E2 | 182-D1106-308 | IgK | SEQ ID NO: 40 | SEQ ID NO: 25 |
| | 175D10 | 182-D1106-362 | IgK | SEQ ID NO: 39 | SEQ ID NO: 24 |
| | 45C1 | 182-D758-187 | IgK | SEQ ID NO: 38 | SEQ ID NO: 23 |
| | 45C1 | 182-D758-187 | IgK | SEQ ID NO: 41 | SEQ ID NO: 26 |
| | 45C1 | 182-D758-187 | IgK | SEQ ID NO: 42 | SEQ ID NO: 27 |
| | 45C1 | 182-D758-187 | IgK | SEQ ID NO: 43 | SEQ ID NO: 28 |

In preferred embodiments, antibodies, in particular chimerised forms of antibodies according to the invention include antibodies comprising a heavy chain constant region (CH) comprising an amino acid sequence derived from a human heavy chain constant region such as the amino acid sequence represented by SEQ ID NO: 13 or 52, or a functional variant thereof, or a fragment of the amino acid sequence or functional variant. In further preferred embodiments, antibodies, in particular chimerised forms of antibodies according to the invention include antibodies comprising a light chain constant region (CL) comprising an amino acid sequence derived from a human light chain constant region such as the amino acid sequence represented by SEQ ID NO: 12 or a functional variant thereof, or a fragment of the amino acid sequence or functional variant. In a particular preferred embodiment, antibodies, in particular chimerised forms of antibodies according to the invention include antibodies which comprise a CH comprising an amino acid sequence derived from a human CH such as the amino acid sequence represented by SEQ ID NO: 13 or 52, or a functional variant thereof, or a fragment of the amino acid sequence or functional variant and which comprise a CL comprising an amino acid sequence derived from a human CL such as the amino acid sequence represented by SEQ ID NO: 12 or a functional variant thereof, or a fragment of the amino acid sequence or functional variant.

In one embodiment, an anti-CLDN18.2 antibody is a chimeric mouse/human IgG1 monoclonal antibody comprising kappa, murine variable light chain, human kappa light chain constant region allotype Km(3), murine heavy chain variable region, human IgG1 constant region, allotype G1m(3).

In certain preferred embodiments, chimerised forms of antibodies include antibodies comprising a heavy chain comprising an amino acid sequence selected from the group consisting of SEQ ID NOs: 14, 15, 16, 17, 18, 19, 51, and a functional variant thereof, or a fragment of the amino acid sequence or functional variant and/or comprising a light chain comprising an amino acid sequence selected from the group consisting of SEQ ID NOs: 20, 21, 22, 23, 24, 25, 26, 27, 28, and a functional variant thereof, or a fragment of the amino acid sequence or functional variant.

In certain preferred embodiments, chimerised forms of antibodies include antibodies comprising a combination of heavy chains and light chains selected from the following possibilities (i) to (ix):
(i) the heavy chain comprises an amino acid sequence represented by SEQ ID NO: 14 or a functional variant thereof, or a fragment of the amino acid sequence or functional variant and the light chain comprises an amino acid sequence represented by SEQ ID NO: 21 or a functional variant thereof, or a fragment of the amino acid sequence or functional variant,
(ii) the heavy chain comprises an amino acid sequence represented by SEQ ID NO: 15 or a functional variant thereof, or a fragment of the amino acid sequence or functional variant and the light chain comprises an amino acid sequence represented by SEQ ID NO: 20 or a functional variant thereof, or a fragment of the amino acid sequence or functional variant,
(iii) the heavy chain comprises an amino acid sequence represented by SEQ ID NO: 16 or a functional variant thereof, or a fragment of the amino acid sequence or functional variant and the light chain comprises an amino acid sequence represented by SEQ ID NO: 22 or a functional variant thereof, or a fragment of the amino acid sequence or functional variant,
(iv) the heavy chain comprises an amino acid sequence represented by SEQ ID NO: 18 or a functional variant thereof, or a fragment of the amino acid sequence or functional variant and the light chain comprises an amino acid sequence represented by SEQ ID NO: 25 or a functional variant thereof, or a fragment of the amino acid sequence or functional variant,
(v) the heavy chain comprises an amino acid sequence represented by SEQ ID NO: 17 or a functional variant thereof, or a fragment of the amino acid sequence or functional variant and the light chain comprises an amino acid sequence represented by SEQ ID NO: 24 or a functional variant thereof, or a fragment of the amino acid sequence or functional variant,
(vi) the heavy chain comprises an amino acid sequence represented by SEQ ID NO: 19 or a functional variant thereof, or a fragment of the amino acid sequence or functional variant and the light chain comprises an amino acid sequence represented by SEQ ID NO: 23 or a functional variant thereof, or a fragment of the amino acid sequence or functional variant,
(vii) the heavy chain comprises an amino acid sequence represented by SEQ ID NO: 19 or a functional variant thereof, or a fragment of the amino acid sequence or functional variant and the light chain comprises an amino acid sequence represented by SEQ ID NO: 26 or a functional variant thereof, or a fragment of the amino acid sequence or functional variant,
(viii) the heavy chain comprises an amino acid sequence represented by SEQ ID NO: 19 or a functional variant thereof, or a fragment of the amino acid sequence or functional variant and the light chain comprises an amino acid sequence represented by SEQ ID NO: 27 or a functional variant thereof, or a fragment of the amino acid sequence or functional variant,
(ix) the heavy chain comprises an amino acid sequence represented by SEQ ID NO: 19 or a functional variant thereof, or a fragment of the amino acid sequence or functional variant and the light chain comprises an amino acid sequence represented by SEQ ID NO: 28 or a functional variant thereof, or a fragment of the amino acid sequence or functional variant, and
(x) the heavy chain comprises an amino acid sequence represented by SEQ ID NO: 51 or a functional variant thereof, or a fragment of the amino acid sequence or functional variant and the light chain comprises an amino acid sequence represented by SEQ ID NO: 24 or a functional variant thereof, or a fragment of the amino acid sequence or functional variant.

In one particularly preferred embodiment, an anti-CLDN18.2 antibody comprises a heavy chain comprising an amino acid sequence represented by SEQ ID NO: 17 or a functional variant thereof, or a fragment of the amino acid sequence or functional variant and a light chain comprising an amino acid sequence represented by SEQ ID NO: 24 or a functional variant thereof, or a fragment of the amino acid sequence or functional variant.

In one particularly preferred embodiment, an anti-CLDN18.2 antibody comprises a heavy chain comprising an amino acid sequence represented by SEQ ID NO: 51 or a functional variant thereof, or a fragment of the amino acid sequence or functional variant and a light chain comprising an amino acid sequence represented by SEQ ID NO: 24 or a functional variant thereof, or a fragment of the amino acid sequence or functional variant.

A fragment of an amino acid sequence selected from the group consisting of SEQ ID NOs: 14, 15, 16, 17, 18, 19, 51, 20, 21, 22, 23, 24, 25, 26, 27, and 28 preferably relates to said sequence wherein 17, 18, 19, 20, 21, 22 or 23 amino acids at the N-terminus are removed.

In a preferred embodiment, an anti-CLDN18.2 antibody comprises a heavy chain variable region (VH) comprising an amino acid sequence selected from the group consisting of SEQ ID NOs: 29, 30, 31, 32, 33, 34, and a functional variant thereof, or a fragment of the amino acid sequence or functional variant.

In a preferred embodiment, an anti-CLDN18.2 antibody comprises a light chain variable region (VL) comprising an amino acid sequence selected from the group consisting of SEQ ID NO: 35, 36, 37, 38, 39, 40, 41, 42, 43, and a functional variant thereof, or a fragment of the amino acid sequence or functional variant.

In certain preferred embodiments, an anti-CLDN18.2 antibody comprises a combination of heavy chain variable region (VH) and light chain variable region (VL) selected from the following possibilities (i) to (ix):
(i) the VH comprises an amino acid sequence represented by SEQ ID NO: 29 or a functional variant thereof, or a fragment of the amino acid sequence or functional variant and the VL comprises an amino acid sequence represented by SEQ ID NO: 36 or a functional variant thereof, or a fragment of the amino acid sequence or functional variant,
(ii) the VH comprises an amino acid sequence represented by SEQ ID NO: 30 or a functional variant thereof, or a fragment of the amino acid sequence or functional variant and the VL comprises an amino acid sequence represented by SEQ ID NO: 35 or a functional variant thereof, or a fragment of the amino acid sequence or functional variant,
(iii) the VH comprises an amino acid sequence represented by SEQ ID NO: 31 or a functional variant thereof, or a fragment of the amino acid sequence or functional variant and the VL comprises an amino acid sequence represented by SEQ ID NO: 37 or a functional variant thereof, or a fragment of the amino acid sequence or functional variant,
(iv) the VH comprises an amino acid sequence represented by SEQ ID NO: 33 or a functional variant thereof, or a fragment of the amino acid sequence or functional variant and the VL comprises an amino acid sequence represented by SEQ ID NO: 40 or a functional variant thereof, or a fragment of the amino acid sequence or functional variant,
(v) the VH comprises an amino acid sequence represented by SEQ ID NO: 32 or a functional variant thereof, or a fragment of the amino acid sequence or functional variant and the VL comprises an amino acid sequence represented by SEQ ID NO: 39 or a functional variant thereof, or a fragment of the amino acid sequence or functional variant,
(vi) the VH comprises an amino acid sequence represented by SEQ ID NO: 34 or a functional variant thereof, or a fragment of the amino acid sequence or functional variant and the VL comprises an amino acid sequence represented by SEQ ID NO: 38 or a functional variant thereof, or a fragment of the amino acid sequence or functional variant,
(vii) the VH comprises an amino acid sequence represented by SEQ ID NO: 34 or a functional variant thereof, or a fragment of the amino acid sequence or functional variant and the VL comprises an amino acid sequence represented by SEQ ID NO: 41 or a functional variant thereof, or a fragment of the amino acid sequence or functional variant,
(viii) the VH comprises an amino acid sequence represented by SEQ ID NO: 34 or a functional variant thereof, or a fragment of the amino acid sequence or functional variant and the VL comprises an amino acid sequence represented by SEQ ID NO: 42 or a functional variant thereof, or a fragment of the amino acid sequence or functional variant,
(ix) the VH comprises an amino acid sequence represented by SEQ ID NO: 34 or a functional variant thereof, or a fragment of the amino acid sequence or functional variant and the VL comprises an amino acid sequence represented by SEQ ID NO: 43 or a functional variant thereof, or a fragment of the amino acid sequence or functional variant.

In one particularly preferred embodiment, an anti-CLDN18.2 antibody comprises a VH comprising an amino acid sequence represented by SEQ ID NO: 32 or a functional variant thereof, or a fragment of the amino acid sequence or functional variant and a VL comprising an amino acid sequence represented by SEQ ID NO: 39 or a functional variant thereof, or a fragment of the amino acid sequence or functional variant. In a more preferred embodiment, the anti-CLDN18.2 antibody comprises a VH comprising an amino acid sequence represented by SEQ ID NO: 32 and a VL comprises an amino acid sequence represented by SEQ ID NO: 39, such as IMAB362 (Zolbetuximab).

The term “fragment” refers, in particular, to one or more of the complementarity-determining regions (CDRs), preferably at least the CDR3 sequence, optionally in combination with the CDR1 sequence and/or the CDR2 sequence, of the heavy chain variable region (VH) and/or of the light chain variable region (VL). In one embodiment said one or more of the complementarity-determining regions (CDRs) are selected from a set of complementarity-determining regions CDR1, CDR2 and CDR3. In a particularly preferred embodiment, the term “fragment” refers to the complementarity-determining regions CDR1, CDR2 and CDR3 of the heavy chain variable region (VH) and/or of the light chain variable region (VL).

In a preferred embodiment, an anti-CLDN18.2 antibody comprises a VH comprising a set of complementarity-determining regions CDR1, CDR2 and CDR3 selected from the following embodiments (i) to (vi):
(i) CDR1: positions 45-52 of SEQ ID NO: 14, CDR2: positions 70-77 of SEQ ID NO: 14, CDR3: positions 116-125 of SEQ ID NO: 14,
(ii) CDR1: positions 45-52 of SEQ ID NO: 15, CDR2: positions 70-77 of SEQ ID NO: 15, CDR3: positions 116-126 of SEQ ID NO: 15,
(iii) CDR1: positions 45-52 of SEQ ID NO: 16, CDR2: positions 70-77 of SEQ ID NO: 16, CDR3: positions 116-124 of SEQ ID NO: 16,
(iv) CDR1: positions 45-52 of SEQ ID NO: 17, CDR2: positions 70-77 of SEQ ID NO: 17, CDR3: positions 116-126 of SEQ ID NO: 17,
(v) CDR1: positions 44-51 of SEQ ID NO: 18, CDR2: positions 69-76 of SEQ ID NO: 18, CDR3: positions 115-125 of SEQ ID NO: 18, and
(vi) CDR1: positions 45-53 of SEQ ID NO: 19, CDR2: positions 71-78 of SEQ ID NO: 19, CDR3: positions 117-128 of SEQ ID NO: 19.

In a preferred embodiment, an anti-CLDN18.2 antibody comprises a VH comprising at least one, preferably two, more preferably all three of the CDR sequences of a set of complementarity-determining regions CDR1, CDR2 and CDR3 selected from the above embodiments (i) to (vi).

In a preferred embodiment, an anti-CLDN18.2 antibody comprises a VL comprising a set of complementarity-determining regions CDR1, CDR2 and CDR3 selected from the following embodiments (i) to (ix):
(i) CDR1: positions 47-58 of SEQ ID NO: 20, CDR2: positions 76-78 of SEQ ID NO: 20, CDR3: positions 115-123 of SEQ ID NO: 20,
(ii) CDR1: positions 49-53 of SEQ ID NO: 21, CDR2: positions 71-73 of SEQ ID NO: 21, CDR3: positions 110-118 of SEQ ID NO: 21,
(iii) CDR1: positions 47-52 of SEQ ID NO: 22, CDR2: positions 70-72 of SEQ ID NO: 22, CDR3: positions 109-117 of SEQ ID NO: 22,
(iv) CDR1: positions 47-58 of SEQ ID NO: 23, CDR2: positions 76-78 of SEQ ID NO: 23, CDR3: positions 115-123 of SEQ ID NO: 23,
(v) CDR1: positions 47-58 of SEQ ID NO: 24, CDR2: positions 76-78 of SEQ ID NO: 24, CDR3: positions 115-123 of SEQ ID NO: 24,
(vi) CDR1: positions 47-58 of SEQ ID NO: 25, CDR2: positions 76-78 of SEQ ID NO: 25, CDR3: positions 115-122 of SEQ ID NO: 25,
(vii) CDR1: positions 47-58 of SEQ ID NO: 26, CDR2: positions 76-78 of SEQ ID NO: 26, CDR3: positions 115-123 of SEQ ID NO: 26,
(viii) CDR1: positions 47-58 of SEQ ID NO: 27, CDR2: positions 76-78 of SEQ ID NO: 27, CDR3: positions 115-123 of SEQ ID NO: 27, and
(ix) CDR1: positions 47-52 of SEQ ID NO: 28, CDR2: positions 70-72 of SEQ ID NO: 28, CDR3: positions 109-117 of SEQ ID NO: 28.

In a preferred embodiment, an anti-CLDN18.2 antibody comprises a VL comprising at least one, preferably two, more preferably all three of the CDR sequences of a set of complementarity-determining regions CDR1, CDR2 and CDR3 selected from the above embodiments (i) to (ix).

In a preferred embodiment, an anti-CLDN18.2 antibody comprises a combination of VH and VL each comprising a set of complementarity-determining regions CDR1, CDR2 and CDR3 selected from the following embodiments (i) to (ix):
(i) VH: CDR1: positions 45-52 of SEQ ID NO: 14, CDR2: positions 70-77 of SEQ ID NO: 14, CDR3: positions 116-125 of SEQ ID NO: 14, VL: CDR1: positions 49-53 of SEQ ID NO: 21, CDR2: positions 71-73 of SEQ ID NO: 21, CDR3: positions 110-118 of SEQ ID NO: 21,
(ii) VH: CDR1: positions 45-52 of SEQ ID NO: 15, CDR2: positions 70-77 of SEQ ID NO: 15, CDR3: positions 116-126 of SEQ ID NO: 15, VL: CDR1: positions 47-58 of SEQ ID NO: 20, CDR2: positions 76-78 of SEQ ID NO: 20, CDR3: positions 115-123 of SEQ ID NO: 20,
(iii) VH: CDR1: positions 45-52 of SEQ ID NO: 16, CDR2: positions 70-77 of SEQ ID NO: 16, CDR3: positions 116-124 of SEQ ID NO: 16, VL: CDR1: positions 47-52 of SEQ ID NO: 22, CDR2: positions 70-72 of SEQ ID NO: 22, CDR3: positions 109-117 of SEQ ID NO: 22,
(iv) VH: CDR1: positions 44-51 of SEQ ID NO: 18, CDR2: positions 69-76 of SEQ ID NO: 18, CDR3: positions 115-125 of SEQ ID NO: 18, VL: CDR1: positions 47-58 of SEQ ID NO: 25, CDR2: positions 76-78 of SEQ ID NO: 25, CDR3: positions 115-122 of SEQ ID NO: 25,
(v) VH: CDR1: positions 45-52 of SEQ ID NO: 17, CDR2: positions 70-77 of SEQ ID NO: 17, CDR3: positions 116-126 of SEQ ID NO: 17, VL: CDR1: positions 47-58 of SEQ ID NO: 24, CDR2: positions 76-78 of SEQ ID NO: 24, CDR3: positions 115-123 of SEQ ID NO: 24,
(vi) VH: CDR1: positions 45-53 of SEQ ID NO: 19, CDR2: positions 71-78 of SEQ ID NO: 19, CDR3: positions 117-128 of SEQ ID NO: 19, VL: CDR1: positions 47-58 of SEQ ID NO: 23, CDR2: positions 76-78 of SEQ ID NO: 23, CDR3: positions 115-123 of SEQ ID NO: 23,
(vii) VH: CDR1: positions 45-53 of SEQ ID NO: 19, CDR2: positions 71-78 of SEQ ID NO: 19, CDR3: positions 117-128 of SEQ ID NO: 19, VL: CDR1: positions 47-58 of SEQ ID NO: 26, CDR2: positions 76-78 of SEQ ID NO: 26, CDR3: positions 115-123 of SEQ ID NO: 26,
(viii) VH: CDR1: positions 45-53 of SEQ ID NO: 19, CDR2: positions 71-78 of SEQ ID NO: 19, CDR3: positions 117-128 of SEQ ID NO: 19, VL: CDR1: positions 47-58 of SEQ ID NO: 27, CDR2: positions 76-78 of SEQ ID NO: 27, CDR3: positions 115-123 of SEQ ID NO: 27, and
(ix) VH: CDR1: positions 45-53 of SEQ ID NO: 19, CDR2: positions 71-78 of SEQ ID NO: 19, CDR3: positions 117-128 of SEQ ID NO: 19, VL: CDR1: positions 47-52 of SEQ ID NO: 28, CDR2: positions 70-72 of SEQ ID NO: 28, CDR3: positions 109-117 of SEQ ID NO: 28.

In a preferred embodiment, an anti-CLDN18.2 antibody comprises a VH comprising at least one, preferably two, more preferably all three of the VH CDR sequences of a set of complementarity-determining regions CDR1, CDR2 and CDR3 selected from the above embodiments (i) to (ix) and a VL comprising at least one, preferably two, more preferably all three of the VL CDR sequences of a set of complementarity-determining regions CDR1, CDR2 and CDR3 from the same embodiment (i) to (ix).

The term “at least one, preferably two, more preferably all three of the CDR sequences” preferably relates to at least the CDR3 sequence, optionally in combination with the CDR1 sequence and/or the CDR2 sequence.

In one particularly preferred embodiment, an anti-CLDN18.2 antibody comprises a combination of VH and VL each comprising a set of complementarity-determining regions CDR1, CDR2 and CDR3 as follows:

VH: CDR1: positions 45-52 of SEQ ID NO: 17, CDR2: positions 70-77 of SEQ ID NO: 17, CDR3: positions 116-126 of SEQ ID NO: 17, VL: CDR1: positions 47-58 of SEQ ID NO: 24, CDR2: positions 76-78 of SEQ ID NO: 24, CDR3: positions 115-123 of SEQ ID NO: 24.

In further preferred embodiments, an anti-CLDN18.2 antibody preferably comprises one or more of the complementarity-determining regions (CDRs), preferably at least the CDR3 variable region, of the heavy chain variable region (VH) and/or of the light chain variable region (VL) of a monoclonal antibody against CLDN18.2, preferably of a monoclonal antibody against CLDN18.2 described herein, and preferably comprises one or more of the complementarity-determining regions (CDRs), preferably at least the CDR3 variable region, of the heavy chain variable regions (VH) and/or light chain variable regions (VL) described herein. In one embodiment said one or more of the complementarity-determining regions (CDRs) are selected from a set of complementarity-determining regions CDR1, CDR2 and CDR3 described herein. In a particularly preferred embodiment, an anti-CLDN18.2 antibody preferably comprises the complementarity-determining regions CDR1, CDR2 and CDR3 of the heavy chain variable region (VH) and/or of the light chain variable region (VL) of a monoclonal antibody against CLDN18.2, preferably of a monoclonal antibody against CLDN18.2 described herein, and preferably comprises the complementarity-determining regions CDR1, CDR2 and CDR3 of the heavy chain variable regions (VH) and/or light chain variable regions (VL) described herein.

In one embodiment an antibody comprising one or more CDRs, a set of CDRs or a combination of sets of CDRs as described herein comprises said CDRs together with their intervening framework regions. Preferably, the portion will also include at least about 50% of either or both of the first and fourth framework regions, the 50% being the C-terminal 50% of the first framework region and the N-terminal 50% of the fourth framework region. Construction of antibodies made by recombinant DNA techniques may result in the introduction of residues N- or C-terminal to the variable regions encoded by linkers introduced to facilitate cloning or other manipulation steps, including the introduction of linkers to join variable regions or join variable regions to further protein sequences including sequences as described herein.

In one embodiment an antibody comprising one or more CDRs, a set of CDRs or a combination of sets of CDRs as described herein comprises said CDRs in a human antibody framework.

Reference herein to an antibody comprising with respect to the heavy chain thereof a particular chain, or a particular region or sequence preferably relates to the situation wherein all heavy chains of said antibody comprise said particular chain, region or sequence. This applies correspondingly to the light chain of an antibody.

It is possible that the anti-CLDN18.2 antibodies described herein (e.g., expressed by different cell lines) have different glycosylation patterns. However, all anti-CLDN18.2 antibodies are considered described herein, regardless of their glycosylation pattern or modification or deletion thereof. Thus, for the purposes of the present disclosure, anti-CLDN18.2 antibodies can be glycosylated or non-glycosylated. When the anti-CLDN18.2 antibodies are glycosylated, they can have any possible glycosylation pattern. In addition, each heavy chain in an antibody can have the same glycosylation pattern or the two heavy chains can have different glycosylation patterns. Also described herein is site-directed mutagenesis of the CH2 domain of antibody to eliminate glycosylation, to avoid changes in the immunogenicity, pharmacokinetics and/or effector functions resulting from non-human glycosylation.

As used herein, the term “glycosylation” means the pattern of carbohydrate units that are covalently linked with an antibody. When it is said that the anti-CLDN18.2 antibodies described herein have a particular glycosylation pattern, it is understood that most of the referenced anti-CLDN18.2 antibodies have that particular glycosylation pattern. In other aspects, when it is said that the anti-CLDN18.2 antibodies described herein have a particular glycosylation pattern, it is understood that a number greater than or equal to 50, 75, 90, 95, 99 or 100% of the antibodies have that particular glycosylation pattern.

The glycosylation of polypeptides is typically N-linked or O-linked. The glycosylation of antibody polypeptides is typically N-linked and forms a biantennary structure. N-linked refers to the connection of the carbohydrate moiety with the side chain of an asparagine residue. The tripeptide sequences asparagine-X-serine, asparagine-X-threonine, and asparagine-X-cysteine, in which X is any amino acid except proline, are the recognition sequences for the enzymatic connection of the carbohydrate moiety with the asparagine side chain. Therefore, the presence of any of these tripeptide sequences in an antibody creates a potential glycosylation site.

Three different biantennary glycan structures designated “G0”, “G1” and “G2” have 0, 1 or 2, respectively, terminal galactose residues at the non-reducing end of glycan. In some cases, the glycan structure may also have a fucose residue bound to an N-acetylglucosamine, which is covalently linked to the amino acid asparagine in the antibody. When fucose (F) is present, the biantennary glycan nomenclature is changed to “GOF”, “GIF” or “G2F”, depending on the number of terminal galactose residues. In addition, when the antibody contains both heavy chains, the glycan nomenclature is repeated for each of the two heavy chains. The glycoform “GOF, GOF” is a species in which both heavy chains have the glycan G0 connected and each glycan G0 has a fucose residue (F) bound with a N-acetylglucosamine. The glycoform “GOF, GIF” is a species in which one of the heavy chains has the glycan G0 connected and the other heavy chain has the glycan G1 connected, each glycan G0 and glycan G1 having a fucose residue (F) linked to an N-acetylglucosamine.

In various embodiments, the anti-CLDN18.2 antibodies described herein have a glycosylation pattern that is predominantly GOF or GIF, in particular GOF. Anti-CLDN18.2 antibodies can have a glycosylation pattern that is GOF for more than 50%, more than 60%, more than 70% or even higher. Anti-CLDN18.2 antibodies can have a glycosylation pattern that is GOF for 65% to 80%. Anti-CLDN18.2 antibodies can have a glycosylation pattern that is GIF for 10% to 20%.

In various embodiments, the anti-CLDN18.2 antibodies described herein have a glycosylation pattern that can be selected from the group consisting of “GOF, GOF”, “GOF, GIF”, and “GIF, GIF”, and mixtures thereof. Anti-CLDN18.2 antibodies can have a glycosylation pattern that is “GOF, GOF” for more than 50% of the antibodies produced. Anti-CLDN18.2 antibodies can have a glycosylation pattern that is “GOF, GIF” for less than 50% of the antibodies produced. For example, the anti-CLDN18.2 antibodies described herein may have a glycosylation pattern that is “GOF, GOF” or “GOF, GIF”. Anti-CLDN18.2 antibodies may have a mixture of different glycosylation patterns. For example, anti-CLDN18.2 antibodies may be a mixture of antibodies in which some have a glycosylation pattern “GOF, GOF” and others have a glycosylation pattern “GOF, GIF”, e.g., at a ratio of about 1:1, 1.5:1, 2:1, 3:1, 4:1, 5:1, 6:1, 7:1, or even higher.

In one embodiment, an anti-CLDN18.2 antibody competes for CLDN18.2 binding with an anti-CLDN18.2 antibody described herein and/or has the specificity for CLDN18.2 of an anti-CLDN18.2 antibody described herein. In these and other embodiments, an anti-CLDN18.2 antibody may be highly homologous to an anti-CLDN18.2 antibody described herein. It is contemplated that a preferred anti-CLDN18.2 antibody has CDR regions either identical or highly homologous to the CDR regions of an anti-CLDN18.2 antibody described herein. By “highly homologous” it is contemplated that from 1 to 5, preferably from 1 to 4, such as 1 to 3 or 1 or 2 substitutions may be made in each CDR region.

The term “compete” refers to the competition between two binding molecules, e.g., antibodies, for binding to a target antigen. If two binding molecules do not block each other for binding to a target antigen, such binding molecules are non-competing and this is an indication that said binding molecules do not bind to the same part, i.e., epitope, of the target antigen. It is well known to a person skilled in the art how to test for competition of binding molecules such as antibodies for binding to a target antigen. An example of such a method is a so-called cross-competition assay, which may, e.g., be performed as an ELISA or by flow-cytometry. For example, an ELISA-based assay may be performed by coating ELISA plate wells with one of the antibodies; adding the competing antibody and His-tagged antigen/target and detecting whether the added antibody inhibited binding of the His-tagged antigen to the coated antibody, e.g., by adding biotinylated anti-His antibody, followed by Streptavidin-poly-HRP, and further developing the reaction with ABTS and measuring the absorbance at 405 nm. For example, a flow-cytometry assay may be performed by incubating cells expressing the antigen/target with an excess of unlabeled antibody, incubating the cells with a sub-optimal concentration of biotin-labelled antibody, followed by incubation with fluorescently labeled streptavidin and analyzing by flow cytometry.

Two binding molecules have the “same specificity” if they bind to the same antigen and to the same epitope. Whether a molecule to be tested recognizes the same epitope as a certain binding molecule, i.e., the binding molecules bind to the same epitope, can be tested by different methods known to the skilled person. The competition of the binding molecules such as antibodies for the same epitope may provide an indication for the binding molecules binding to the same epitope. The competition between the binding molecules can be detected by a cross-blocking assay. For example, a competitive ELISA assay may be used as a cross-blocking assay. For example, target antigen may be coated on the wells of a microtiter plate and antigen binding antibody and candidate competing test antibody may be added. The amount of the antigen binding antibody bound to the antigen in the well indirectly correlates with the binding ability of the candidate competing test antibody that competes therewith for binding to the same epitope. Specifically, the larger the affinity of the candidate competing test antibody is for the same epitope, the smaller the amount of the antigen binding antibody bound to the antigen-coated well. The amount of the antigen binding antibody bound to the well can be measured by labeling the antibody with detectable or measurable labeling substances.

“Homologous” refers to the sequence similarity or sequence identity between two polypeptides or between two nucleic acid molecules. When a position in both of the two compared sequences is occupied by the same base or amino acid monomer subunit, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. Generally, a comparison is made when two sequences are aligned to give maximum homology. Homologous sequences exhibit according to the disclosure at least 40%, in particular at least 50%, at least 60%, at least 70%, at least 80%, at least 90% and preferably at least 95%, at least 98 or at least 99% identity of the amino acid or nucleotide residues.

“Fragment”, with reference to an amino acid sequence (peptide or protein), relates to a part of an amino acid sequence, i.e., a sequence which represents the amino acid sequence shortened at the N-terminus and/or C-terminus. A fragment shortened at the C-terminus (N-terminal fragment) is obtainable e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90%, at least 95%, at least 96%, at least 97%, at least 98%, or at least 99% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

A “fragment” of an antibody sequence, when it replaces said antibody sequence in an antibody, preferably retains binding of said antibody to CLDN18.2 and preferably functions of said antibody as described herein, e.g., CDC mediated lysis or ADCC mediated lysis.

By “variant” or “variant protein” or “variant polypeptide” herein is meant a protein that differs from a parent protein by virtue of at least one amino acid modification. The parent polypeptide may be a naturally occurring or wild type (WT) polypeptide, or may be a modified version of a wild type polypeptide. Preferably, the variant polypeptide has at least one amino acid modification compared to the parent polypeptide, e.g., from 1 to about 20 amino acid modifications, and preferably from 1 to about 10 or from 1 to about 5 amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild type polypeptide, or a variant or engineered version of a wild type polypeptide.

By “wild type” or “WT” or “native” herein is meant an amino acid sequence that is found in nature, including allelic variations. A wild type protein or polypeptide has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant of said given amino acid sequence will be at least about 60%, 65%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS:needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences.

The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two sequences being compared, dividing this number by the number of positions compared and multiplying the result obtained by 100 so as to obtain the percentage identity between these two sequences.

The teaching given herein with respect to specific amino acid sequences, e.g., those shown in the sequence listing, is to be construed so as to also relate to variants of said specific sequences resulting in sequences which are functionally equivalent to said specific sequences, e.g., amino acid sequences exhibiting properties identical or similar to those of the specific amino acid sequences. One important property is to retain binding of an antibody to its target or to sustain effector functions of an antibody. Preferably, a sequence which is a variant with respect to a specific sequence, when it replaces the specific sequence in an antibody retains binding of said antibody to CLDN18.2 and preferably functions of said antibody as described herein, e.g., CDC mediated lysis or ADCC mediated lysis.

It will be appreciated by those skilled in the art that in particular the sequences of the CDR, hypervariable and variable regions can be modified without losing the ability to bind CLDN18.2. For example, CDR regions will be either identical or highly homologous to the regions of antibodies specified herein. By “highly homologous” it is contemplated that from 1 to 5, preferably from 1 to 4, such as 1 to 3 or 1 or 2 substitutions may be made in the CDRs. In addition, the hypervariable and variable regions may be modified so that they show substantial homology with the regions of antibodies specifically disclosed herein.

The term “functional variant”, as used herein, refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., binding to a target molecule or contributing to binding to a target molecule. If the parent molecule or sequence is an antibody molecule or sequence, the alteration is preferably not in the variable regions of the antibody, more preferably not in the CDR regions of the antibody. In one embodiment, a functional variant either alone or in combination with other elements competes for binding to a target molecule with the parent molecule or sequence. In other words, the modifications in the amino acid sequence of the parent molecule or sequence do not significantly affect or alter the binding characteristics of the molecule or sequence. In different embodiments, binding of the functional variant may be reduced but still significantly present, e.g., binding of the functional variant may be at least 50%, at least 60%, at least 70%, at least 80%, or at least 90% of the parent molecule or sequence. However, in other embodiments, binding of the functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof.

The term “nucleic acid”, as used herein, is intended to include DNA and RNA. A nucleic acid may be single-stranded or double-stranded, preferably is double-stranded DNA.

According to the invention, the term “expression” is used in its most general meaning and comprises the production of RNA or of RNA and protein/peptide. It also comprises partial expression of nucleic acids. Furthermore, expression may be carried out transiently or stably.

The term “transgenic animal” refers to an animal having a genome comprising one or more transgenes, preferably heavy and/or light chain transgenes, or transchromosomes (either integrated or non-integrated into the animal's natural genomic DNA) and which is preferably capable of expressing the transgenes. For example, a transgenic mouse can have a human light chain transgene and either a human heavy chain transgene or human heavy chain transchromosome, such that the mouse produces human anti-CLDN18.2 antibodies when immunized with CLDN18.2 antigen and/or cells expressing CLDN18.2. The human heavy chain transgene can be integrated into the chromosomal DNA of the mouse, as is the case for transgenic mice, e.g., HuMAb mice, such as HCo7 or HCo12 mice, or the human heavy chain transgene can be maintained extrachromosomally, as is the case for transchromosomal (e.g., KM) mice as described in WO 02/43478. Such transgenic and transchromosomal mice may be capable of producing multiple isotypes of human monoclonal antibodies to CLDN18.2 (e.g., IgG, IgA and/or IgE) by undergoing V-D-J recombination and isotype switching.

“Reduce”, “decrease” or “inhibit” as used herein means an overall decrease or the ability to cause an overall decrease, preferably of 5% or greater, 10% or greater, 20% or greater, more preferably of 50% or greater, and most preferably of 75% or greater, in the level, e.g., in the level of expression or in the level of proliferation of cells.

The term “inhibition of tumor growth” with respect to a particular treatment means reduction in tumor size caused by the treatment, e.g., when compared to the non-treated tumor or compared to a control treatment. This term includes a decrease, i.e., retardation, of tumor growth, a reduction in tumor size compared to the size of the tumor at the start of treatment, i.e., tumor regression, and a complete disappearance of a tumor, i.e., complete remission. In one embodiment, tumor regression, e.g., as expressed by tumor regression rate, caused by the treatment described herein is 5% or more, 10% or more, 15% or more, 20% or more, 25% or more, 30% or more or even higher.

Terms such as “increase” or “enhance” preferably relate to an increase or enhancement by about at least 10%, preferably at least 20%, preferably at least 30%, more preferably at least 40%, more preferably at least 50%, even more preferably at least 80%, and most preferably at least 100%, at least 200%, at least 500%, at least 1000%, at least 10000% or even more.

“Inducing” when used in relation to a certain activity or function such as antibody-dependent cell-mediated cytotoxicity (ADCC) may mean that there was no such activity or function present before induction, but it may also mean that there was a certain level of such activity or function present before induction and after induction said activity or function is enhanced. Thus, the term “inducing” also includes “enhancing”.

### Mechanisms of mAb Action

Although the following provides considerations regarding the mechanism underlying the therapeutic efficacy of antibodies of the invention it is not to be considered as limiting to the invention in any way.

The antibodies described herein preferably interact with components of the immune system, preferably through ADCC or CDC. Antibodies described herein can also be used to target payloads (e.g., radioisotopes, drugs or toxins) to directly kill tumor cells or can be used with traditional chemotherapeutic agents, attacking tumors through complementary mechanisms of action that may include anti-tumor immune responses that may have been compromised owing to a chemotherapeutic's cytotoxic side effects on T lymphocytes. However, antibodies described herein may also exert an effect simply by binding to CLDN18.2 on the cell surface, thus, e.g., blocking proliferation of the cells.

### Antibody-Dependent Cell-Mediated Cytotoxicity

ADCC describes the cell-killing ability of effector cells as described herein, in particular lymphocytes, which preferably requires the target cell being marked by an antibody.

ADCC preferably occurs when antibodies bind to antigens on tumor cells and the antibody Fc domains engage Fc receptors (FcR) on the surface of immune effector cells. Several families of Fc receptors have been identified, and specific cell populations characteristically express defined Fc receptors. ADCC can be viewed as a mechanism to directly induce a variable degree of immediate tumor destruction that leads to antigen presentation and the induction of tumor-directed NK cell or T-cell responses. Preferably, in vivo induction of ADCC will lead to tumor-directed T-cell responses and host-derived antibody responses.

### Complement-Dependent Cytotoxicity

CDC is another cell-killing method that can be directed by antibodies. IgM is the most effective isotype for complement activation. IgG1 and IgG3 are also both very effective at directing CDC via the classical complement-activation pathway. Preferably, in this cascade, the formation of antigen-antibody complexes results in the uncloaking of multiple C1q binding sites in close proximity on the CH2 domains of participating antibody molecules such as IgG molecules (C1q is one of three subcomponents of complement C1). Preferably these uncloaked C1q binding sites convert the previously low-affinity C1q-IgG interaction to one of high avidity, which triggers a cascade of events involving a series of other complement proteins and leads to the proteolytic release of the effector-cell chemotactic/activating agents C3a and C5a. Preferably, the complement cascade ends in the formation of a membrane attack complex, which creates pores in the cell membrane that facilitate free passage of water and solutes into and out of the cell.

Antibodies described herein can be produced by a variety of techniques, including conventional monoclonal antibody methodology, e.g., the standard somatic cell hybridization technique of Kohler and Milstein, Nature 256: 495 (1975). Although somatic cell hybridization procedures are preferred, in principle, other techniques for producing monoclonal antibodies can be employed, e.g., viral or oncogenic transformation of B-lymphocytes or phage display techniques using libraries of antibody genes.

The preferred animal system for preparing hybridomas that secrete monoclonal antibodies is the murine system. Hybridoma production in the mouse is a very well established procedure. Immunization protocols and techniques for isolation of immunized splenocytes for fusion are known in the art. Fusion partners (e.g., murine myeloma cells) and fusion procedures are also known.

Other preferred animal systems for preparing hybridomas that secrete monoclonal antibodies are the rat and the rabbit system (e.g., described in Spieker-Polet et al., Proc. Natl. Acad. Sci. U.S.A. 92:9348 (1995), see also Rossi et al., Am. J. Clin. Pathol. 124: 295 (2005)).

In yet another preferred embodiment, human monoclonal antibodies can be generated using transgenic or transchromosomal mice carrying parts of the human immune system rather than the mouse system. These transgenic and transchromosomic mice include mice known as HuMAb mice and KM mice, respectively, and are collectively referred to herein as “transgenic mice.” The production of human antibodies in such transgenic mice can be performed as described in detail for CD20 in WO2004 035607

Yet another strategy for generating monoclonal antibodies is to directly isolate genes encoding antibodies from lymphocytes producing antibodies of defined specificity, e.g., see Babcock et al., 1996; A novel strategy for generating monoclonal antibodies from single, isolated lymphocytes producing antibodies of defined specificities. For details of recombinant antibody engineering see also Welschof and Kraus, Recombinant antibodies for cancer therapy ISBN-0-89603-918-8 and Benny K. C. Lo Antibody Engineering ISBN 1-58829-092-1.

To generate antibodies, mice can be immunized with carrier-conjugated peptides derived from the antigen sequence, i.e., the sequence against which the antibodies are to be directed, an enriched preparation of recombinantly expressed antigen or fragments thereof and/or cells expressing the antigen, as described. Alternatively, mice can be immunized with DNA encoding the antigen or fragments thereof. In the event that immunizations using a purified or enriched preparation of the antigen do not result in antibodies, mice can also be immunized with cells expressing the antigen, e.g., a cell line, to promote immune responses.

The immune response can be monitored over the course of the immunization protocol with plasma and serum samples being obtained by tail vein or retroorbital bleeds. Mice with sufficient titers of immunoglobulin can be used for fusions. Mice can be boosted intraperitonealy or intravenously with antigen expressing cells 3 days before sacrifice and removal of the spleen to increase the rate of specific antibody secreting hybridomas.

To generate hybridomas producing monoclonal antibodies, splenocytes and lymph node cells from immunized mice can be isolated and fused to an appropriate immortalized cell line, such as a mouse myeloma cell line. The resulting hybridomas can then be screened for the production of antigen-specific antibodies. Individual wells can then be screened by ELISA for antibody secreting hybridomas. By Immunofluorescence and FACS analysis using antigen expressing cells, antibodies with specificity for the antigen can be identified. The antibody secreting hybridomas can be replated, screened again, and if still positive for monoclonal antibodies can be subcloned by limiting dilution. The stable subclones can then be cultured in vitro to generate antibody in tissue culture medium for characterization.

Antibodies also can be produced in a host cell transfectoma using, for example, a combination of recombinant DNA techniques and gene transfection methods as are well known in the art (Morrison, S. (1985) Science 229: 1202).

For example, in one embodiment, the gene(s) of interest, e.g., antibody genes, can be ligated into an expression vector such as a eukaryotic expression plasmid such as used by the GS gene expression system disclosed in WO 87/04462, WO 89/01036 and EP 338 841 or other expression systems well known in the art. The purified plasmid with the cloned antibody genes can be introduced in eukaryotic host cells such as CHO cells, NS/0 cells, HEK293T cells or HEK293 cells or alternatively other eukaryotic cells like plant derived cells, fungal or yeast cells. The method used to introduce these genes can be methods described in the art such as electroporation, lipofectine, lipofectamine or others. After introduction of these antibody genes in the host cells, cells expressing the antibody can be identified and selected. These cells represent the transfectomas which can then be amplified for their expression level and upscaled to produce antibodies. Recombinant antibodies can be isolated and purified from these culture supernatants and/or cells.

Alternatively, the cloned antibody genes can be expressed in other expression systems, including prokaryotic cells, such as microorganisms, e.g., E. coli. Furthermore, the antibodies can be produced in transgenic non-human animals, such as in milk from sheep and rabbits or in eggs from hens, or in transgenic plants; see e.g., Verma, R., et al. (1998) J. Immunol. Meth. 216: 165-181; Pollock, et al. (1999) J. Immunol. Meth. 231: 147-157; and Fischer, R., et al. (1999) Biol. Chem. 380: 825-839.

### Chimerization

Murine monoclonal antibodies can be used as therapeutic antibodies in humans when labeled with toxins or radioactive isotopes. Nonlabeled murine antibodies are highly immunogenic in man when repetitively applied leading to reduction of the therapeutic effect. The main immunogenicity is mediated by the heavy chain constant regions. The immunogenicity of murine antibodies in man can be reduced or completely avoided if respective antibodies are chimerized or humanized. Chimeric antibodies are antibodies, the different portions of which are derived from different animal species, such as those having a variable region derived from a murine antibody and a human immunoglobulin constant region. Chimerisation of antibodies is achieved by joining of the variable regions of the murine antibody heavy and light chain with the constant region of human heavy and light chain (e.g., as described by Kraus et al., in Methods in Molecular Biology series, Recombinant antibodies for cancer therapy ISBN-0-89603-918-8). In a preferred embodiment chimeric antibodies are generated by joining human kappa-light chain constant region to murine light chain variable region. In an also preferred embodiment chimeric antibodies can be generated by joining human lambda-light chain constant region to murine light chain variable region. The preferred heavy chain constant regions for generation of chimeric antibodies are IgG1, IgG3 and IgG4. Other preferred heavy chain constant regions for generation of chimeric antibodies are IgG2, IgA, IgD and IgM.

### Humanization

Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332: 323-327; Jones, P. et al. (1986) Nature 321: 522-525; and Queen, C. et al. (1989) Proc. Natl. Acad. Sci. U.S.A. 86: 10029-10033). Such framework sequences can be obtained from public DNA databases that include germline antibody gene sequences. These germline sequences will differ from mature antibody gene sequences because they will not include completely assembled variable genes, which are formed by V (D) J joining during B cell maturation. Germline gene sequences will also differ from the sequences of a high affinity secondary repertoire antibody at individual evenly across the variable region.

The ability of antibodies to bind an antigen can be determined using standard binding assays (e.g., ELISA, Western Blot, Immunofluorescence and flow cytometric analysis).

To purify antibodies, selected hybridomas can be grown in two-liter spinner-flasks for monoclonal antibody purification. Alternatively, antibodies can be produced in dialysis based bioreactors. Supernatants can be filtered and, if necessary, concentrated before affinity chromatography with protein G-sepharose or protein A-sepharose. Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

To determine if the selected monoclonal antibodies bind to unique epitopes, site-directed or multi-site directed mutagenesis can be used.

To determine the isotype of antibodies, isotype ELISAs with various commercial kits (e.g., Zymed, Roche Diagnostics) can be performed. Wells of microtiter plates can be coated with anti-mouse Ig. After blocking, the plates are reacted with monoclonal antibodies or purified isotype controls, at ambient temperature for two hours. The wells can then be reacted with either mouse IgG1, IgG2a, IgG2b or IgG3, IgA or mouse IgM-specific peroxidase-conjugated probes. After washing, the plates can be developed with ABTS substrate (1 mg/ml) and analyzed at OD of 405-650. Alternatively, the IsoStrip Mouse Monoclonal Antibody Isotyping Kit (Roche, Cat. No. 1493027) may be used as described by the manufacturer.

In order to demonstrate presence of antibodies in sera of immunized mice or binding of monoclonal antibodies to living cells expressing antigen, flow cytometry can be used. Cell lines expressing naturally or after transfection antigen and negative controls lacking antigen expression (grown under standard growth conditions) can be mixed with various concentrations of monoclonal antibodies in hybridoma supernatants or in PBS containing 1% FBS, and can be incubated at 4° C. for 30 min. After washing, the APC- or Alexa647-labeled anti IgG antibody can bind to antigen-bound monoclonal antibody under the same conditions as the primary antibody staining. The samples can be analyzed by flow cytometry with a FACS instrument using light and side scatter properties to gate on single, living cells. In order to distinguish antigen-specific monoclonal antibodies from non-specific binders in a single measurement, the method of co-transfection can be employed. Cells transiently transfected with plasmids encoding antigen and a fluorescent marker can be stained as described above. Transfected cells can be detected in a different fluorescence channel than antibody-stained cells. As the majority of transfected cells express both transgenes, antigen-specific monoclonal antibodies bind preferentially to fluorescence marker expressing cells, whereas non-specific antibodies bind in a comparable ratio to non-transfected cells. An alternative assay using fluorescence microscopy may be used in addition to or instead of the flow cytometry assay. Cells can be stained exactly as described above and examined by fluorescence microscopy.

In order to demonstrate presence of antibodies in sera of immunized mice or binding of monoclonal antibodies to living cells expressing antigen, immunofluorescence microscopy analysis can be used. For example, cell lines expressing either spontaneously or after transfection antigen and negative controls lacking antigen expression are grown in chamber slides under standard growth conditions in DMEM/F12 medium, supplemented with 10% fetal calf serum (FCS), 2 mM L-glutamine, 100 IU/ml penicillin and 100 μg/ml streptomycin. Cells can then be fixed with methanol or paraformaldehyde or left untreated. Cells can then be reacted with monoclonal antibodies against the antigen for 30 min. at 25° C. After washing, cells can be reacted with an Alexa555-labelled anti-mouse IgG secondary antibody (Molecular Probes) under the same conditions. Cells can then be examined by fluorescence microscopy.

Cell extracts from cells expressing antigen and appropriate negative controls can be prepared and subjected to sodium dodecyl sulfate (SDS) polyacrylamide gel electrophoresis. After electrophoresis, the separated antigens will be transferred to nitrocellulose membranes, blocked, and probed with the monoclonal antibodies to be tested. IgG binding can be detected using anti-mouse IgG peroxidase and developed with ECL substrate.

Antibodies can be further tested for reactivity with antigen by Immunohistochemistry in a manner well known to the skilled person, e.g., using paraformaldehyde or acetone fixed cryosections or paraffin embedded tissue sections fixed with paraformaldehyde from non-cancer tissue or cancer tissue samples obtained from patients during routine surgical procedures or from mice carrying xenografted tumors inoculated with cell lines expressing spontaneously or after transfection antigen. For immunostaining, antibodies reactive to antigen can be incubated followed by horseradish-peroxidase conjugated goat anti-mouse or goat anti-rabbit antibodies (DAKO) according to the vendors instructions.

Antibodies can be tested for their ability to mediate phagocytosis and killing of cells expressing CLDN18.2. The testing of monoclonal antibody activity in vitro will provide an initial screening prior to testing in vivo models.

### Antibody Dependent Cell-Mediated Cytotoxicity (ADC° C.):

Briefly, polymorphonuclear cells (PMNs), NK cells, monocytes, mononuclear cells or other effector cells, from healthy donors can be purified by Ficoll Hypaque density centrifugation, followed by lysis of contaminating erythrocytes. Washed effector cells can be suspended in RPMI supplemented with 10% heat-inactivated fetal calf serum or, alternatively with 5% heat-inactivated human serum and mixed with 51Cr labeled target cells expressing CLDN18.2, at various ratios of effector cells to target cells. Alternatively, the target cells may be labeled with a fluorescence enhancing ligand (BATDA). A highly fluorescent chelate of Europium with the enhancing ligand which is released from dead cells can be measured by a fluorometer. Another alternative technique may utilize the transfection of target cells with luciferase. Added lucifer yellow may then be oxidated by viable cells only. Purified anti-CLDN18.2 IgGs can then be added at various concentrations. Irrelevant human IgG can be used as negative control. Assays can be carried out for 4 to 20 hours at 37° C. depending on the effector cell type used. Samples can be assayed for cytolysis by measuring 51Cr release or the presence of the EuTDA chelate in the culture supernatant. Alternatively, luminescence resulting from the oxidation of lucifer yellow can be a measure of viable cells.

Anti-CLDN18.2 monoclonal antibodies can also be tested in various combinations to determine whether cytolysis is enhanced with multiple monoclonal antibodies.

### Complement Dependent Cytotoxicity (CDC):

Monoclonal anti-CLDN18.2 antibodies can be tested for their ability to mediate CDC using a variety of known techniques. For example, serum for complement can be obtained from blood in a manner known to the skilled person. To determine the CDC activity of mAbs, different methods can be used. 51Cr release can for example be measured or elevated membrane permeability can be assessed using a propidium iodide (PI) exclusion assay. Briefly, target cells can be washed and 5×10⁵/ml can be incubated with various concentrations of mAb for 10⁻³⁰ min. at room temperature or at 37° C. Serum or plasma can then be added to a final concentration of 20% (v/v) and the cells incubated at 37° C. for 20-30 min. All cells from each sample can be added to the PI solution in a FACS tube. The mixture can then be analyzed immediately by flow cytometry analysis using FACSArray.

In an alternative assay, induction of CDC can be determined on adherent cells. In one embodiment of this assay, cells are seeded 24 h before the assay with a density of 3×10⁴/well in tissue-culture flat-bottom microtiter plates. The next day growth medium is removed and the cells are incubated in triplicates with antibodies. Control cells are incubated with growth medium or growth medium containing 0.2% saponin for the determination of background lysis and maximal lysis, respectively. After incubation for 20 min. at room temperature supernatant is removed and 20% (v/v) human plasma or serum in DMEM (prewarmed to 37° C.) is added to the cells and incubated for another 20 min. at 37° C. All cells from each sample are added to propidium iodide solution (10 μg/ml). Then, supernatants are replaced by PBS containing 2.5 μg/ml ethidium bromide and fluorescence emission upon excitation at 520 nm is measured at 600 nm using a Tecan Safire. The percentage specific lysis is calculated as follows: % specific lysis=(fluorescence sample-fluorescence background)/(fluorescence maximal lysis-fluorescence background)×100.

### Induction of Apoptosis and Inhibition of Cell Proliferation by Monoclonal Antibodies:

To test for the ability to initiate apoptosis, monoclonal anti-CLDN18.2 antibodies can, for example, be incubated with CLDN18.2 positive tumor cells, e.g., SNU-16, DAN-G, KATO-III or CLDN18.2 transfected tumor cells at 37ºC for about 20 hours. The cells can be harvested, washed in Annexin-V binding buffer (BD biosciences), and incubated with Annexin V conjugated with FITC or APC (BD biosciences) for 15 min. in the dark. All cells from each sample can be added to PI solution (10 μg/ml in PBS) in a FACS tube and assessed immediately by flow cytometry (as above). Alternatively, a general inhibition of cell-proliferation by monoclonal antibodies can be detected with commercially available kits. The DELFIA Cell Proliferation Kit (Perkin-Elmer, Cat. No. AD0200) is a non-isotopic immunoassay based on the measurement of 5-bromo-2′-deoxyuridine (BrdU) incorporation during DNA synthesis of proliferating cells in microplates. Incorporated BrdU is detected using europium labelled monoclonal antibody. To allow antibody detection, cells are fixed and DNA denatured using Fix solution. Unbound antibody is washed away and DELFIA inducer is added to dissociate europium ions from the labelled antibody into solution, where they form highly fluorescent chelates with components of the DELFIA Inducer. The fluorescence measured—utilizing time-resolved fluorometry in the detection—is proportional to the DNA synthesis in the cell of each well.

### Preclinical Studies

Monoclonal antibodies which bind to CLDN18.2 also can be tested in an in vivo model (e.g., in immune deficient mice carrying xenografted tumors inoculated with cell lines expressing CLDN18.2, e.g., DAN-G, SNU-16, or KATO-III, or after transfection, e.g., HEK293) to determine their efficacy in controlling growth of CLDN18.2-expressing tumor cells.

In vivo studies after xenografting CLDN18.2 expressing tumor cells into immunocompromised mice or other animals can be performed using antibodies described herein. Antibodies can be administered to tumor free mice followed by injection of tumor cells to measure the effects of the antibodies to prevent formation of tumors or tumor-related symptoms. Antibodies can be administered to tumor-bearing mice to determine the therapeutic efficacy of respective antibodies to reduce tumor growth, metastasis or tumor related symptoms. Antibody application can be combined with application of other substances as immune checkpoint inhibitors, cystostatic drugs, growth factor inhibitors, cell cycle blockers, angiogenesis inhibitors or other antibodies to determine efficacy and potential toxicity of combinations. To analyze toxic side effects mediated by antibodies animals can be inoculated with antibodies or control reagents and thoroughly investigated for symptoms possibly related to CLDN18.2-antibody therapy. Possible side effects of in vivo application of CLDN18.2 antibodies particularly include toxicity at CLDN18.2 expressing tissues including stomach. Antibodies recognizing CLDN18.2 in human and in other species, e.g., mice, are particularly useful to predict potential side effects mediated by application of monoclonal CLDN18.2-antibodies in humans.

Mapping of epitopes recognized by antibodies can be performed as described in detail in “Epitope Mapping Protocols (Methods in Molecular Biology) by Glenn E. Morris ISBN-089603-375-9 and in “Epitope Mapping: A Practical Approach” Practical Approach Series, 248 by Olwyn M. R. Westwood, Frank C. Hay.

The compounds and agents described herein may be administered in the form of any suitable pharmaceutical composition.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

Pharmaceutical compositions are usually provided in a uniform dosage form and may be prepared in a manner known per se. A pharmaceutical composition may e.g., be in the form of a solution or suspension.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, bulking agents, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical compositions is formulated for systemic administration. In another preferred embodiment, the systemic administration is by intravenous administration. The compositions may be injected directly into a tumor or lymph node.

The term “co-administering” as used herein means a process whereby different compounds or compositions are administered to the same patient. For example, the compounds or compositions may be administered simultaneously, at essentially the same time, or sequentially.

The agents and compositions described herein can be administered to patients, e.g., in vivo, to treat or prevent a variety of disorders such as those described herein. Preferred patients include human patients having disorders that can be corrected or ameliorated by administering the agents and compositions described herein. This includes disorders involving cells characterized by expression of CLDN18.2.

For example, in one embodiment, agents described herein can be used to treat a patient with a cancer disease, e.g., a cancer disease such as described herein characterized by the presence of cancer cells expressing CLDN18.2.

The pharmaceutical compositions and methods of treatment described according to the invention may also be used for immunization or vaccination to prevent a disease described herein.

As used herein, an “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

The present invention is further described by the following figures and examples, which are used only for illustration purposes and are not meant to be limiting. Owing to the description and the examples, further embodiments which are likewise included in the invention are accessible to the skilled worker.

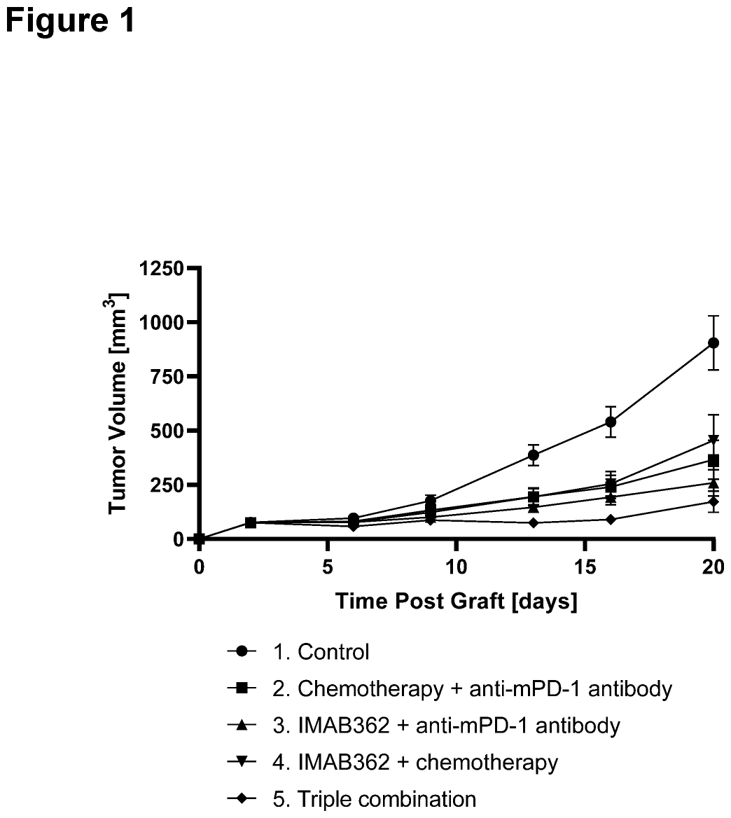

### FIG. 1

Anti-tumor activity of IMAB362 with anti-mPD-1 antibody and chemotherapy in CLS-103 LVT-murinCLDN18.2 gastric cancer syngeneic mouse model. CLS-103 LVT-murinCLDN18.2 gastric carcinoma cells were inoculated (2×10⁶ cells per mouse) into the right flank of female NMRI mice. Inoculated mice were randomized 2 days after tumor inoculation (n=16 per group). Test agents were administered according to the respective administration group. Tumor growth curve of each administration group from day 0 to day 20 is shown (Mean±SEM).

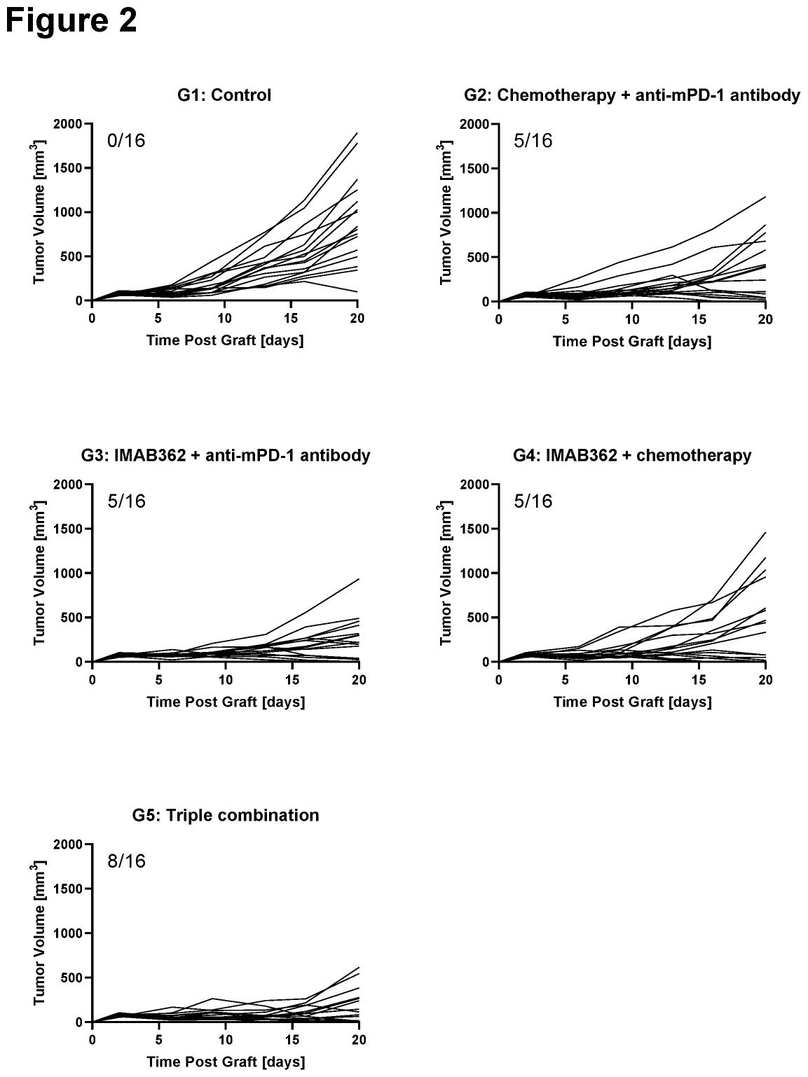

### FIG. 2

Spider plot analysis representing individual tumor growth curves of all treated mice from day 0 to day 20. Upper left: the number of regressed tumors in each treatment group.

### EXAMPLES

### Example 1: Efficacy Studies of the Combination of Anti-CLDN18.2 Antibodies, Chemotherapy and Immune Checkpoint Inhibitors In Vivo

In order to demonstrate that a combination of an anti-CLDN18.2 antibody, chemotherapy and an immune checkpoint inhibitor improves anti-tumor activity over a combination of an anti-CLDN18.2 antibody and chemotherapy, an anti-CLDN18.2 antibody and an immune checkpoint inhibitor or chemotherapy and an immune checkpoint inhibitor in vivo, anti-tumor activity of IMAB362 in combination with chemotherapy and an anti-mPD-1 antibody will be examined up to day 28 or, for endpoint survival, up to day 84 in a subcutaneously transplanted syngeneic model in immunocompetent outbred Crl:NMRI(Han) mice using CLS-103 gastric carcinoma cells with lentiviral transduction of murine CLDN18.2 (CLS-103 LVT-murinCLDN18.2). Rituximab will be used as an isotype control of IMAB362.

### Test Agents

Anti-CLDN18.2 antibody: IMAB362 (Astellas Pharma Inc.)
Control antibody: Rituximab BS Intravenous Infusion [KHK] 500 mg (Kyowa Kirin Co., Ltd., Cat #22900AMX00971000)
Chemotherapy: Oxaliplatin (Yakult Honsha Co. Ltd., Cat #22100AMX02236) and 5-flurouracil (Kyowa Kirin Co., Ltd, Cat #22500AMX00515)
Anti-mPD-1 antibody: In VivoMAb anti-mouse PD-1, clone RMP1-14 (BioXCell, Cat #BE0146)
Isotype control antibody: InVivoMAb rat IgG2a isotype control, anti-trinitrophenol, clone 2A3 (BioXCell, Cat #BE0089)

### CLS-103 LVT-murinCLDN18.2 Gastric Cancer Mouse Model

CLS-103 LVT-murinCLDN18.2 will be engrafted subcutaneously into the right flank of female Crl:NMRI(Han) mice (9 to 12 weeks-old) at 2×10⁶ cells/mouse. Mice will be randomized based on tumor volume measured 2 days after engraftment into groups (n=12 per group). The day of randomization will be defined as day 0. IMAB362 or control antibody Rituximab will be administered at 800 μg/mouse. Anti-mPD-1 antibody or isotype control antibody will be administered at 100 μg/mouse. All antibodies will be administered by intraperitoneal injections twice per week starting on day 0. Oxaliplatin and 5-fluorouracil will be administered by intraperitoneal injection twice per week starting on day 0, with oxaliplatin being administered at 1 mg/kg body weight and 5-fluorouracil being administered at 30 mg/kg body weight. Tumors will be measured twice per week. The study endpoint will be defined as day 84. Tumor volume will be determined by length×width×width×0.5. Tumor growth inhibition (TGI [%]) or tumor regression rate (TRR [%]) of each group will be calculated using the equations described below. Complete regression (CR) will be determined as the tumor volume of individual regressed to zero.

TGI [%]=100×(1−increase of mean tumor volume of each group #÷increase of mean tumor volume of control group #)

#: increase of mean tumor volume [mm³]=mean tumor volume of the last measurement of each group−mean tumor volume at randomization (day 0)

TRR [%]=100×(1−mean tumor volume of the last measurement of each group÷mean tumor volume at randomization of each group)

### Results

In this mouse CLS-103 LVT-murinCLDN18.2 tumor study, IMAB362 in combination with chemotherapy and anti-mPD-1 antibody may improve the anti-tumor effect which is determined by the number of CR or by survival rate up to day 84 in a synergistic manner. As shown in the following table, the number of CR, TGI % (TRR %) and the survival rate are shown for all treatment groups. At primary endpoint, combination treatment comprising 800 μg of IMAB362+1 mg/kg oxaliplatin+30 mg/kg 5-fluorouracil, 800 μg of IMAB362+100 μg of anti-mPD-1 antibody, or 1 mg/kg oxaliplatin+30 mg/kg 5-fluorouracil+100 μg of anti-mPD-1 antibody may result in 6 CR in the group of 12 mice. The treatment group of the combination of IMAB362, chemotherapy and anti-mPD-1 antibody may show an increased number of mice with CR and improved survival compared to that of double agent groups in a synergistic manner in the mouse model. TGI % (TRR %) will be exploratorily evaluated at time point where all animals in all groups will still be present. Treatment with 800 μg of IMAB362+1 mg/kg oxaliplatin+30 mg/kg 5-fluorouracil, 800 μg of IMAB362+100 μg of anti-mPD-1 antibody, or 1 mg/kg oxaliplatin+30 mg/kg 5-fluorouracil+100 μg of anti-mPD-1 antibody may produce 70% to 95% TGI, respectively, whereas the combination treatment comprising 800 μg of IMAB362+1 mg/kg Oxaliplatin+30 mg/kg 5-Fluorouracil+100 μg of anti-mPD-1 antibody may not only inhibit but even regress tumor up to 10% or even more.

| | Primary | Exploratory | |
|---|---|---|---|
| | endpoint: | endpoint: | Exploratory |
| | CR number | TGI % | endpoint: |
| Treatment | (n = 12) | (TRR %) | Survival % |
| Control | 1/12 | — | 0%-10% |
| (Rituximab/Vehicle/Isotype) | | | |
| IMAB362/Chemo | 5-6/12 | 70%-95% | 40%-60% |
| IMAB362/mPD-1 | 5-6/12 | 70%-95% | 40%-60% |
| Chemo/mPD-1 | 5-6/12 | 70%-95% | 40%-60% |
| IMAB362/Chemo/mPD-1 | 9-11/12 | (10%-30%) | 80%-90% |

### Example 2: In Vivo Efficacy Study of the Combination of Anti-CLDN18.2 Antibodies, Chemotherapy and Immune Checkpoint Inhibitors Using CLS-103 LVT-murinCLDN18.2 Gastric Cancer Syngeneic Mouse Model

To evaluate the effect of triple combination therapy, an anti-CLDN18.2 antibody, IMAB362, combined with chemotherapy (5-fluorouracil (5-FU) and oxaliplatin) and immune checkpoint inhibitor was investigated using a syngeneic mouse tumor model bearing CLS-103 mouse gastric carcinoma cells, in which mouse CLDN18.2 was lentivirally transduced (CLS-103 LVT-murinCLDN18.2). CLS-103 LVT-murinCLDN18.2 gastric carcinoma cells were subcutaneously inoculated into immunocompetent Crl:NMRI(Han) mice, and inoculated mice were randomized into 5 groups (n=16) with nearly equal mean tumor volume in each group. Test agents were administered in combinations as listed below. To determine whether the triple combination of anti-CLDN18.2 antibody, chemotherapy and an immune checkpoint inhibitor enhances the anti-tumor efficacy over dual combinations in vivo, tumor growth inhibition was examined up to day 20. Additionally, the numbers of regressed tumors were compared between each treatment group. Rituximab was used as an isotype control of IMAB362. PBS and 5% glucose were used as vehicle of 5-FU and oxaliplatin, respectively.

### Test Agents

Anti-CLDN18.2 antibody: IMAB362 (Astellas Pharma Inc.)
Control antibody: Rituximab BS Intravenous Infusion [KHK] 500 mg (Kyowa Kirin Co., Ltd., Cat #22900AMX00971000)
Chemotherapy: 5-fluorouracil (5-FU) (Kyowa Kirin Co., Ltd., Cat #22500AMX00515), oxaliplatin (Yakult Honsha Co. Ltd., Cat #22100AMX02236)
Anti-mPD-1 antibody: In VivoMAb anti-mouse PD-1, clone RMP1-14 (BioXCell, Cat #BE0146)
Isotype control antibody: InVivoMAb rat IgG2a isotype control, anti-trinitrophenol, clone 2A3 (BioXCell, Cat #BE0089)
Vehicle: PBS as vehicle for 5-FU, 5% glucose as vehicle for oxaliplatin

### Administration Groups

Group 1: Control (control antibody+PBS+5% glucose+isotype control antibody)
Group 2: Dual combination of anti-mPD-1 antibody+chemotherapy (control antibody+5-FU+oxaliplatin+anti-mPD-1 antibody)
Group 3: Dual combination of anti-CLDN18.2 antibody+anti-mPD-1 antibody (IMAB362+PBS+5% glucose+anti-mPD-1 antibody)
Group 4: Dual combination of anti-CLDN18.2 antibody+chemotherapy (IMAB362+5-FU+oxaliplatin+isotype control antibody)
Group 5: Triple combination of anti-CLDN18.2 antibody+anti-mPD-1 antibody+chemotherapy (IMAB362+5-FU+oxaliplatin+anti-mPD-1 antibody)

### CLS-103 LVT-murinCLDN18.2 Gastric Cancer Syngeneic Mouse Model

CLS-103 LVT-murinCLDN18.2 gastric carcinoma cells were engrafted subcutaneously into the right flank of female immunocompetent Crl:NMRI(Han) mice (10-week-old) at 2×10⁶ cells/head. The day of tumor inoculation was defined as day 0. Mice were randomized based on tumor volume measured 2 days after engraftment into 5 groups (n=16 per group). IMAB362 or control antibody, rituximab, was administered at 800 μg/head. Chemotherapy consisted of 5-FU and oxaliplatin administered at 10 mg/kg (3.33 mL/kg) and 0.5 mg/kg (3.33 mL/kg), respectively. Anti-mPD-1 antibody or isotype control antibody was administered at 30 μg/head. All test agents were administered by intraperitoneal injections twice per week starting on day 2. Tumor sizes were measured twice per week. The final measurement point was on day 20. Tumor volume was determined by length×width×width×0.5. Tumor growth inhibition (TGI [%]) of each group was calculated using the equation described below. Tumor regression was determined by the definition described below.

TGI [%]=100×(1−increase of mean tumor volume of each group #÷increase of mean tumor volume of control group #)

#: increase of tumor volume [mm³]=mean tumor volume of the last measurement of each group−mean tumor volume at randomization (day 2)
Regression: tumor volume at the last measurement point is smaller than its initial tumor volume at randomization (day 2)

### Results

In this anti-tumor study using CLS-103 LVT-murinCLDN18.2 gastric cancer syngeneic mouse model, the triple combination of IMAB362 with chemotherapy and anti-mPD-1 antibody demonstrated improved anti-tumor activity when compared to the dual combination groups. On day 20, the triple combination of 800 mg IMAB362+chemotherapy (10 mg/kg 5-FU+0.5 mg/kg oxaliplatin)+anti-mPD-1 antibody resulted in the highest TGI rate of 88% among all treatment groups, whereas the dual combinations of chemotherapy (10 mg/kg 5-FU+0.5 mg/kg oxaliplatin)+30 μg of anti-mPD-1 antibody, 800 μg IMAB362+30 μg of anti-mPD-1 antibody, or 800 μg IMAB362+chemotherapy (10 mg/kg 5-FU+0.5 mg/kg oxaliplatin) resulted in TGI rates of 65%, 78%, and 54%, respectively. A spider plot analysis representing individual tumor growth curves of all treated mice demonstrated a marked delay of tumor growth in the triple combination group. In addition, the number of regressed tumors was determined in each treatment group. The triple combination treatment resulted in tumor regression in 8 out of 16 treated mice, compared to 5 out of 16 in the dual combination treatment groups and 0 out of 16 in the control group.

### Tumor Growth Inhibition and Regression of Each Treatment Group

| | | Regression |
|---|---|---|
| Groups | TGI (%) | (n = 16) |
| 1. Control | — | 0 |
| 2. Chemotherapy + anti-mPD-1 antibody | 65 | 5 |
| 3. IMAB362 + anti-mPD-1 antibody | 78 | 5 |
| 4. IMAB362 + chemotherapy | 54 | 5 |
| 5. Triple combination | 88 | 8 |

TGI: tumor growth inhibition; regression: the number of regressed tumors out of a pool of 16 mice.

1. A method for treating or preventing cancer in a patient, comprising administering to the patient an anti-CLDN18.2 antibody, a platinum compound, a fluoropyrimidine compound or precursor thereof and an immune checkpoint inhibitor selected from a PD-1 inhibitor and a PD-L1 inhibitor.

2. A method for inhibiting growth of a tumor in a patient having cancer, comprising administering to the patient an anti-CLDN18.2 antibody, a platinum compound, a fluoropyrimidine compound or precursor thereof and an immune checkpoint inhibitor selected from a PD-1 inhibitor and a PD-L1 inhibitor.

3. The method of claim 1 or 2, wherein the platinum compound is oxaliplatin.

4. The method of any one of claims 1 to 3, wherein the fluoropyrimidine compound or precursor thereof is selected from the group consisting of fluorouracil (5-FU), capecitabine, floxuridine, tegafur, doxifluridine, and carmofur.

5. The method of any one of claims 1 to 4, wherein the fluoropyrimidine compound or precursor thereof is fluorouracil (5-FU) or capecitabine.

6. The method of any one of claims 1 to 5, which comprises administration of oxaliplatin and 5-fluorouracil or a precursor thereof.

7. The method of any one of claims 1 to 6, which comprises administration of oxaliplatin and 5-fluorouracil or oxaliplatin and capecitabine.

8. The method of any one of claims 1 to 7, which comprises administration of folinic acid.

9. The method of any one of claims 1 to 8, which comprises administration of a mFOLFOX6 chemotherapy regimen.

10. The method of any one of claims 1 to 9, wherein the immune checkpoint inhibitor is selected from an anti-PD-1 antibody and an anti-PD-L1 antibody.

11. The method of any one of claims 1 to 10, wherein the immune checkpoint inhibitor is an anti-PD-1 antibody.

12. The method of claim 11, wherein the anti-PD-1 antibody is nivolumab (OPDIVO; BMS-936558), pembrolizumab (KEYTRUDA; MK-3475), pidilizumab (CT-011), cemiplimab (LIBTAYO, REGN2810), spartalizumab (PDR001), MEDI0680 (AMP-514), dostarlimab (TSR-042), cetrelimab (JNJ 63723283), toripalimab (JS001), AMP-224 (GSK-2661380), PF-06801591, tislelizumab (BGB-A317), ABBV-181, BI 754091, or SHR-1210.

13. The method of any one of claims 1 to 10, wherein the immune checkpoint inhibitor is an anti-PD-L1 antibody.

14. The method of claim 13, wherein the anti-PD-L1 antibody is atezolizumab (TECENTRIQ; RG7446; MPDL3280A; R05541267), durvalumab (MEDI4736), BMS-936559, avelumab (bavencio), lodapolimab (LY3300054), CX-072 (Proclaim-CX-072), FAZ053, KN035, or MDX-1105.

15. The method of any one of claims 1 to 12, which comprises administration of oxaliplatin, 5-fluorouracil, folinic acid and nivolumab.

16. The method of any one of claims 1 to 15, wherein the anti-CLDN18.2 antibody binds to the first extracellular loop of CLDN18.2.

17. The method of any one of claims 1 to 16, wherein the anti-CLDN18.2 antibody mediates cell killing by one or more of complement-dependent cytotoxicity (CDC) mediated lysis, antibody-dependent cell-mediated cytotoxicity (ADCC) mediated lysis, induction of apoptosis and inhibition of proliferation.

18. The method of any one of claims 1 to 17, wherein the anti-CLDN18.2 antibody is an antibody selected from the group consisting of:
(i) an antibody produced by and/or obtainable from a clone deposited under the accession no. DSM ACC2737, DSM ACC2738, DSM ACC2739, DSM ACC2740, DSM ACC2741, DSM ACC2742, DSM ACC2743, DSM ACC2745, DSM ACC2746, DSM ACC2747, DSM ACC2748, DSM ACC2808, DSM ACC2809, or DSM ACC2810,
(ii) an antibody which is a chimerized or humanized form of the antibody under (i),
(iii) an antibody having the specificity of the antibody under (i), and
(iv) an antibody comprising the antigen binding portion or antigen binding site, in particular the variable region, of the antibody under (i) and preferably having the specificity of the antibody under (i).

19. The method of any one of claims 1 to 18, wherein the anti-CLDN18.2 antibody comprises a heavy chain variable region CDR1 comprising the sequence of positions 45-52 of the sequence set forth in SEQ ID NO: 17, a heavy chain variable region CDR2 comprising the sequence of positions 70-77 of the sequence set forth in SEQ ID NO: 17, a heavy chain variable region CDR3 comprising the sequence of positions 116-126 of the sequence set forth in SEQ ID NO: 17, a light chain variable region CDR1 comprising the sequence of positions 47-58 of the sequence set forth in SEQ ID NO: 24, a light chain variable region CDR2 comprising the sequence of positions 76-78 of the sequence set forth in SEQ ID NO: 24, and a light chain variable region CDR3 comprising the sequence of positions 115-123 of the sequence set forth in SEQ ID NO: 24.

20. The method of any one of claims 1 to 19, wherein the anti-CLDN18.2 antibody comprises a heavy chain variable region comprising the sequence set forth in SEQ ID NO: 32 or a functional variant thereof, or a fragment of the amino acid sequence or functional variant.

21. The method of any one of claims 1 to 20, wherein the anti-CLDN18.2 antibody comprises a light chain variable region comprising the sequence set forth in SEQ ID NO: 39 or a functional variant thereof, or a fragment of the amino acid sequence or functional variant.

22. The method of any one of claims 1 to 21, wherein the anti-CLDN18.2 antibody comprises a heavy chain constant region comprising the sequence set forth in SEQ ID NO: 13 or 52, or a functional variant thereof, or a fragment of the amino acid sequence or functional variant.

23. The method of any one of claims 1 to 22, wherein the anti-CLDN18.2 antibody comprises a heavy chain comprising the sequence set forth in SEQ ID NO: 17 or 51, or a functional variant thereof, or a fragment of the amino acid sequence or functional variant.

24. The method of any one of claims 1 to 23, wherein the anti-CLDN18.2 antibody comprises a light chain comprising the sequence set forth in SEQ ID NO: 24 or a functional variant thereof, or a fragment of the amino acid sequence or functional variant.

25. The method of any one of claims 1 to 24, wherein the method comprises administering the anti-CLDN18.2 antibody at a dose of up to 1000 mg/m2.

26. The method of any one of claims 1 to 25, wherein the method comprises administering the anti-CLDN18.2 antibody repeatedly at a dose of 300 to 600 mg/m2.

27. The method of any one of claims 1 to 26, wherein the cancer is CLDN18.2 positive.

28. The method of any one of claims 1 to 27, wherein the cancer is an adenocarcinoma, in particular an advanced adenocarcinoma.

29. The method of any one of claims 1 to 28, wherein the cancer is selected from the group consisting of cancer of the stomach, cancer of the esophagus, in particular the lower esophagus, cancer of the eso-gastric junction and gastroesophageal cancer.

30. The method of any one of claims 1 to 29, wherein the cancer is metastatic or locally advanced CLDN18.2-positive, HER2-negative adenocarcinoma of the stomach and eso-gastric junction.

31. The method of any one of claims 1 to 30, wherein CLDN18.2 has the amino acid sequence according to SEQ ID NO: 1.

32. A medical preparation comprising an anti-CLDN18.2 antibody, a platinum compound, a fluoropyrimidine compound or precursor thereof and an immune checkpoint inhibitor selected from a PD-1 inhibitor and a PD-L1 inhibitor.

33. The medical preparation of claim 32, further including printed instructions for use of the preparation for treatment of cancer.
